(19) United States
(12) Patent Application Publication
(10) Pub. No.: US 2026/0207450 A1
(43) Pub. Date: Jul. 23, 2026

(54) ANTI-DANDRUFF COMPOSITIONS

(71) Applicant: Firmenich SA, Satigny (CH)

(72) Inventors: Yang HUANG, Shanghai (CN); Xue-Fei BAI, Shanghai (CN); Ying ZHENG, Shanghai (CN)

(73) Assignee: Firmenich SA, Satigny (CH)

(21) Appl. No.: 19/141,749

(22) PCT Filed: Dec. 21, 2023
(86) PCT No.: PCT/EP2023/087215

§ 371 Date: Jun. 20, 2025

(30) Foreign Application Priority Data
Dec. 21, 2022 (WO) PCT/CN2022/140764
Feb. 2, 2023 (EP) 23154597.1

(51) Int. Cl. A61K 8/34; A61K 8/368; A61K 8/37; A61K 8/49; A61K 8/9794; A61Q 5/00; A61Q 5/02; A61Q 5/12; A61Q 13/00

(52) U.S. Cl. CPC A61K 8/342; A61K 8/368; A61K 8/37; A61K 8/4926; A61K 8/9794; A61Q 5/006; A61Q 5/02; A61Q 5/12; A61Q 13/00; A61K 2800/805

(57) ABSTRACT

The present invention relates to anti-dandruff compositions comprising one or more perfume compounds. The invention also relates to hair care products comprising such compositions as well as methods of treating dandruff using such compositions or products. The invention further relates to a method of identifying compositions useful for the treatment and/or prevention of dandruff.

### INTRODUCTION

Dandruff is a common condition that can leave the scalp inflamed and itchy. This can cause white flakes dusted across the hair. The condition affects approximately 50% of the adult population. While it is not contagious and is not a serious condition from a medical perspective, the appearance of dandruff is undesirable and can cause distress and embarrassment to sufferers.

A major cause of dandruff is the overgrowth of certain species of fungus, in particular Malassezia species. This fungus exists on most adults' scalps. It feeds on the oils on your scalp, breaking it down and leaving oleic acid in its place. Many people are sensitive to oleic acid. The body then reacts to this acid by increasing the speed at which skin cells renew. It is an attempt to rid the scalp of the irritant and can cause flakes. The flakes are dead skin cells that visibly accumulate on the scalp or even shoulders.

A common treatment for dandruff is the use of the compound Zinc Pyrithione (ZPT). However, at least in Europe, this compound has recently been classified as carcinogenic, mutagenic, or toxic for reproduction in cosmetic products, and hence the use of ZPT in cosmetic products is prohibited. In light of this, there is a need to identify alternative anti-dandruff agents which can be used in consumer products. Furthermore, an additional need is that such agents have acceptable hedonic profiles to aid in their wider adoption.

The present inventors sought to identify compounds or compositions which can be used as an anti-dandruff composition.

### DESCRIPTION OF THE FIGURE

FIG. 1: Change in dandruff score (ASFS) after 14 days of two groups treated with Placebo and 0.6% Mix 7, respectively.

### SUMMARY OF THE INVENTION

The present invention relates to an anti-dandruff composition (in the following also abbreviated as ADC) comprising one or more perfume compounds from the following:
(A) a compound of formula (I)

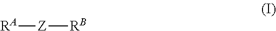

wherein
R^{A} is R₁, wherein R₁ is C₄-C₁₂ alkyl, C₄-C₅ alkenyl, or C₄-C₅ alkynyl; or

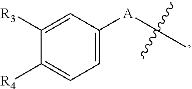

wherein R₃ is H, C₁-C₆ alkyl, or C₁-C₆ alkoxy; R₄ is H, —OH, C₁-C₆ alkyl, or C₁-C₆ alkoxy; and A is a divalent radical selected from the group consisting of a bond, C₁-C₆ alkylene, or C₂-C₆ alkenylene;
Z is C(═O) or CH₂;
R^{B} is H, OH, or OR₂, wherein R₂ is C₁₋₆ alkyl or (carbocyclyl)alkyl; and/or
(B) (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1SR)-3,3-dimethylcyclohexyl]ethoxy}-2-oxoethyl propionate, 2-{(1RS)-1-[(1IRS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2 h-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal.

In a particular embodiment, the compound of formula (I) is selected from one or more of the following:
(i) a compound of formula (II)

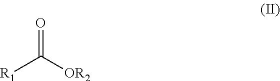

wherein
R₁ is C₄-C₅ alkyl, C₄-C₅ alkenyl, or C₄-C₅ alkynyl, and
R₂ is C₁₋₆ alkyl or (carbocyclyl)alkyl;
(ii) a compound of formula (III)

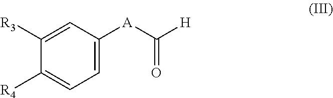

wherein
R₃ is H, C₁-C₆ alkyl, or C₁-C₆ alkoxy;
R₄ is H, OH, C₁-C₆ alkyl optionally substituted by a hydroxy group, or C₁-C₆ alkoxy;
A is a divalent radical selected from the group consisting of a bond, C₁-C₆ alkylene, or C₂-C₆ alkenylene;
(iii) a compound of formula (IV)

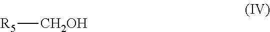

wherein R₅ is C₅-C₁₂ alkyl;
(iv) (2E,6Z)-2,6-nonadienal.

In a particular embodiment, the compound of formula (II) is selected from: methyl octine carbonate, cyclopropylmethyl 3-hexenoate and (+/−)-ethyl 2-methylbutanoate.

In a particular embodiment, the compound of formula (III) is selected from: para-anisyl propanal, cyclamen propanal, (4E)-4-methyl-5-(4-methylphenyl)-4-pentenal, (4E)-2,4-dimethyl-5-(4-methylphenyl)-4-pentenal, cyclamen aldehyde, floral butanal, lilial, ethyl vanillin, 3-[4-(2-hydroxy-2-methylpropyl)phenyl]propanal, and para-anisaldehyde.

In a particular embodiment, the compound of formula (IV) is selected from: 1-decanol, 1-dodecanol, 1-hexanol, and 2-ethyl-1-hexanol.

In a particular embodiment, the anti-dandruff composition further comprises one or more of the following compounds: p-menthan-7-ol, gamma-dodecalactone, methyl dihydrojasmonate, cinnamaldehyde, citral, lemongrass, cinnamon oil, delta-undecalactone, litsea cubeba oil, and 10-undecenal.

In a particular embodiment, the anti-dandruff composition comprises: 1-dodecanol and 1-decanol.

In a particular embodiment, the anti-dandruff composition comprises: 1-dodecanol and p-menthan-7-ol.

In a particular embodiment, the anti-dandruff composition comprises: 1-decanol, p-menthan-7-ol, gamma-dodecalactone, methyl dihydrojasmonate, and geranium rose oil.

In a particular embodiment, the anti-dandruff composition comprises: 1-dodecanol, 1-decanol, cinnamaldehyde, and citral.

In a particular embodiment, the anti-dandruff composition comprises: cinnamaldehyde, citral, 10-undecenal, p-menthan-7-ol, and cyclopropylmethyl 3-hexenoate In a particular embodiment, the anti-dandruff composition comprises: lemongrass, cinnamon oil, and cyclopropylmethyl 3-hexenoate.

In a particular embodiment, the anti-dandruff composition comprises: (2E,6Z)-2,6-nonadienal, cinnamon oil, delta-undecalactone, and litsea cubeba oil.

In a particular embodiment, the anti-dandruff composition further comprises one or more anti-dandruff agents, preferably selected from the group consisting of hexamidine diisethionate, dichlorobenzyl alcohol (DCBA), piroctone olamine, climbazole, ketoconazole, selenium sulphide, coal tar, azelaic acid, salicylic acid, undecylenic acid, zinc pyrithione and mixtures thereof.

An aspect of the invention provides a hair care product comprising an anti-dandruff composition as defined above.

In a particular embodiment, the hair care product is selected from the group comprised of: leave-on hair lotions & sprays, creams, wash-off shampoos, dry shampoos, conditioners, scalp lotions, scalp serums, scalp scrubs, and shower gels.

Another aspect of the present invention relates to the use of an anti-dandruff composition or a hair care product as defined above for the treatment and/or prevention of dandruff.

In a particular embodiment, the use relates to a cosmetic use for the treatment and/or prevention of dandruff, i.e. to a non-therapeutical use.

In another aspect, the present invention relates to an anti-dandruff composition or a hair care product as defined above for use in the treatment and/or prevention of dandruff.

In another aspect, the present invention relates to a method of treatment and/or prevention of dandruff, the method comprising the application to a person in need thereof an anti-dandruff composition or a hair care product as defined above.

In another aspect, the present invention provides a method of identifying a test compound or composition having use in treatment and/or prevention of dandruff, comprising
(a) assaying whether the test composition reduces Malassezia furfur growth, and/or,
(b) assaying whether the test composition reduces Malassezia globosa growth, and/or,
(c) assaying whether the test composition reduces Malassezia restricta growth,
wherein a compound or composition which is positive in one or more assays is of use in/as anti-dandruff compositions.

In a particular embodiment, the method also comprises assaying whether the test composition reduces Staphylococcus aureus growth.

The present invention also relates to the use of
(A) a compound of formula (I)

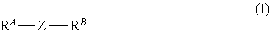

wherein
R^{A} is R₁, wherein R₁ is C₄-C₁₂ alkyl, C₄-C₅ alkenyl, or C₄-C₅ alkynyl; or

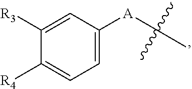

wherein R₃ is H, C₁-C₆ alkyl, or C₁-C₆ alkoxy; R₄ is H, —OH, C₁-C₆ alkyl, or C₁-C₆ alkoxy; and A is a divalent radical selected from the group consisting of a bond, C₁-C₆ alkylene, or C₂-C₆ alkenylene;
Z is C(═O) or CH₂;
R^{B} is H, OH, or OR₂, wherein R₂ is C₁₋₆ alkyl or (carbocyclyl)alkyl; and/or
(B) a compound selected from the group consisting of (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1 SR)-3,3-dimethylcyclohexyl]ethoxy}-2-oxoethyl propionate, 2-{(1 RS)-1-[(1RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2 h-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal,
   for the treatment and/or prevention of dandruff.

The present invention also relates to a method of treatment or prevention of dandruff, the method comprising the application to a person in need thereof a
(A) a compound of formula (I)

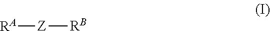

wherein
R^{A} is R₁, wherein R₁ is C₄-C₁₂ alkyl, C₄-C₈ alkenyl, or C₄-C₈ alkynyl; or

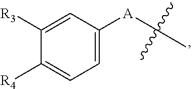

wherein R₃ is H, C₁-C₆ alkyl, or C₁-C₆ alkoxy; R₄ is H, —OH, C₁-C₆ alkyl, or C₁-C₆ alkoxy; and A is a divalent radical selected from the group consisting of a bond, C₁-C₆ alkylene, or C₂-C₆ alkenylene;
Z is C(═O) or CH₂;
R^{B} is H, OH, or OR₂, wherein R₂ is C₁₋₆ alkyl or (carbocyclyl)alkyl; and/or
(B) a compound selected from the group consisting of (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1 SR)-3,3-dimethylcyclohexyl]ethoxy}-2-oxoethyl propionate, 2-{(1 RS)-1-[(1RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2 h-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal.

### DESCRIPTION OF THE INVENTION

### Definitions

Throughout the present disclosure, various chemical names and structures may be recited. Unless otherwise stated, any stereoisomers, such as enantiomers, diastereomers, anomers, epimers, and the like, and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures (+/−), scalemic mixtures, or in pure form. Geometric isomers refer to isomers in which the spatial relationship of atoms around a double bond are different, typically designated E or Z according to conventional understanding in the chemical art. Geometric isomers may each exist in pure form or as mixtures of E and Z isomers. All of the aforementioned isomeric variations of the chemical names or structures recited herein are envisaged.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, 1-methylprop-1-yl, 2-ethylhex-1-yl, decyl, dodecyl, and the like. Unless indicated to the contrary, the term “alkyl” refers to a group that is not further substituted.

As used herein, “alkoxy” means a moiety of the formula —OR wherein R is an alkyl, as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” means a straight or branched hydrocarbon chain containing one or more double bonds. In some embodiments, the alkenyl group has from 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl”, “C₂-C₄ alkenyl”, or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, buta-1,2-dien-4-yl, and pent-2-en-1-yl. Typical alkenyl groups include, but are in no way limited to, ethenyl, propenyl, butenyl, pentenyl, and hexenyl, and the like. Unless indicated to the contrary, the term “alkenyl” refers to a group that is not further substituted.

As used herein, “alkynyl” means a straight or branched hydrocarbon chain containing one or more triple bonds. In some embodiments, the alkynyl group has from 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl”, “C₂-C₄ alkynyl”, or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, 2-butynyl, non-1-yn-1-yl, and the like. Typical alkynyl groups include, but are in no way limited to, ethynyl, propynyl, butynyl, pentynyl, and hexynyl, and the like. Unless indicated to the contrary, the term “alkynyl” refers to a group that is not further substituted.

As used herein, “alkylene” means a branched or straight chain fully saturated di-radical chemical group containing only carbon and hydrogen that is attached to the rest of the molecule via two points of attachment (i.e., an alkanediyl). In some embodiments, the alkylene group has from 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene. Unless indicated to the contrary, the term “alkylene” refers to a group that is not further substituted.

As used herein, “alkenylene” means a straight or branched chain di-radical chemical group containing only carbon and hydrogen and containing at least one carbon-carbon double bond that is attached to the rest of the molecule via two points of attachment. In some embodiments, the alkenylene group has from 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene”, “C₂-C₄ alkenylene”, or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, 2-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 3-methylbut-3-en-1,4-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl. Unless indicated to the contrary, the term “alkenylene” refers to a group that is not further substituted.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. In some embodiments, the carbocyclyl group has from 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl”, “C₃-C₆ carbocyclyl”, or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. Unless indicated to the contrary, the term “carbocyclyl” refers to a group that is not further substituted.

As used herein, “(carbocyclyl)alkyl” means a carbocyclyl group connected, as a substituent, via an alkylene group, such as “C₄₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

### Compositions of the Invention

The present invention relates to anti-dandruff compositions.

As set out herein, given ZPT has recently been classified as carcinogenic, mutagenic, or toxic for reproduction in cosmetic products, and hence the use of ZPT in cosmetic products is prohibited, there is a need to identify alternative anti-dandruff agents which can be used in consumer products. Furthermore, an additional need is that such agents have acceptable hedonic profiles to aid in their wider adoption.

The present inventors sought to identify compounds, which can be used in an anti-dandruff composition. To identify such compounds, they developed a series of assays disclosed in the present application, which can identify such compounds or compositions on the basis that they are able to inhibit the growth of microbial species, which are known to be primary contributors to the cause of dandruff. These compounds can contribute to the overall efficacy of anti-dandruff hair care/scalp care products via their antimicrobial effect alone and/or boosting the effect of anti-dandruff actives, such as hexamidine diisethionate, dichlorobenzyl alcohol (DCBA), piroctone olamine, climbazole, selenium sulphide, coal tar, azelaic acid, ketoconazole, salicylic acid, undecylenic acid, and zinc pyrithione, in particular climbazole, piroctone olamine, hexamidine diisethionate, dichlorobenzyl alcohol (DCBA), salicylic acid and ketoconazole.

They devised a method of identifying whether a test compound or composition can have use in an anti-dandruff composition by: (i) assaying whether the test composition reduces Malassezia furfur growth, (ii) assaying whether the test composition reduces Malassezia globosa growth, and/or (iii) assaying whether the test composition reduces Malassezia restricta growth. Further information concerning the performance of the assays can be identified in the accompanying examples and further described below.

A positive result in one or more of these assays means that the compound or composition can be of use in an anti-dandruff composition.

In a particular embodiment, the above-mentioned assays are disc-diffusion assays.

As used herein by “dandruff” we include a scalp condition that produces white flakes that may be shed and fall from the hair.

An “anti-dandruff” composition is a composition that is capable of reducing or inhibiting the growth of one or more of Malassezia furfur, Malassezia globosa and Malassezia restricta. An anti-dandruff composition is identified by showing a positive result in one or more of the assays as described herein. By “positive result” the inventors mean that the composition inhibits or reduces the growth of one or more of Malassezia furfur, Malassezia globosa and Malassezia restricta, as compared to a control which includes the same sample mixture but lacking the test compound/composition.

Hence, a first aspect of the invention provides an anti-dandruff composition that comprises one or more of the following perfume compounds:
(A) A compound of formula (I)

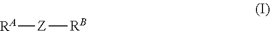

wherein
R^{A} is R₁, wherein R₁ is C₄-C₁₂ alkyl, C₄-C₅ alkenyl, or C₄-C₅ alkynyl; or

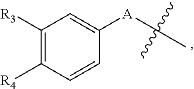

wherein R₃ is H, C₁-C₆ alkyl, or C₁-C₆ alkoxy; R₄ is H, —OH, C₁-C₆ alkyl, or C₁-C₆ alkoxy; and A is a divalent radical selected from the group consisting of a bond, C₁-C₆ alkylene, or C₂-C₆ alkenylene;
Z is C(═O) or CH₂;
R^{B} is H, OH, or OR₂, wherein R₂ is C₁₋₆ alkyl or (carbocyclyl)alkyl; and/or
(B) (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1 SR)-3,3-dimethylcyclohexyl]ethoxy}-2-oxoethyl propionate, 2-{(1 RS)-1-[(1RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2 h-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal.

To the best of the knowledge of the inventors, this is the first-time anti-dandruff compositions have been prepared using one or more compounds as given above.

In a particular embodiment, the C₄-C₅ alkenyl in relation to R₁ in formula (I) is C₆-C₅ alkenyl.

In a particular embodiment, C₁-C₆ alkylene in relation to A in formula (I) is C₂-C₆ alkylene, particularly, C₂-C₄ alkylene, even more particularly, C₂-C₃ alkylene.

In a particular embodiment, C₂-C₆ alkenylene in relation to A in formula (I) is C₃-C₆ alkenylene, particularly, C₄-C₆ alkenylene, even more particularly a C₅-C₆ alkenylene.

In a particular embodiment, C₁₋₆ alkyl in relation to R₂ in formula (I) is C₁ alkyl or C₃₋₆ alkyl, particularly a C₃₋₅ alkyl, even more particularly a C₃₋₄.

In a particular embodiment, C₁₋₆ alkyl in relation to R₂ in formula (I) is C₃₋₆ alkyl.

In a particular embodiment, C₁₋₆ alkyl in relation to R₂ in formula (I) is a methyl, an ethyl or a cyclopropylmethyl group.

In a particular embodiment, C₄-C₁₂ alkyl in relation to R₁ in formula (I) is C₄-C₅, C₇, or C₉ alkyl.

In a particular embodiment, C₁-C₆ alkoxy in relation to R₃ in formula (I) is H or C₃-C₆ alkyl or C₁ or C₃-C₆ alkoxy.

In a particular embodiment, R₄ in formula (I) is H, —OH, or C₁-C₆ alkyl or C₁ alkoxy (methoxy).

In a particular embodiment, C₄-C₁₂ alkyl in relation to R₁ in formula (I) is C₄-C₁₀ alkyl or C₁₂ alkyl.

In a preferred embodiment, the compound of formula (I) is a selected from one or more of the following:
(i) a compound of formula (II)

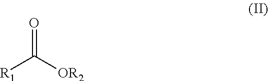

wherein
R₁ is C₄-C₅ alkyl, C₄-C₅ alkenyl, or C₄-C₅ alkynyl, and
R₂ is C₁₋₂ alkyl or (carbocyclyl)alkyl;
(ii) a compound of formula (III)

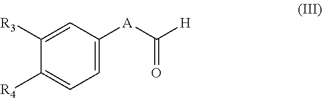

wherein
R₃ is H, C₁-C₆ alkyl, or C₁-C₆ alkoxy;
R₄ is H, OH, C₁-C₆ alkyl optionally substituted by a hydroxy group, or C₁-C₆ alkoxy;
A is a divalent radical selected from the group consisting of a bond, C₁-C₆ alkylene, or C₂-C₆ alkenylene;
(iii) a compound of formula (IV)

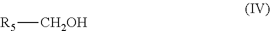

wherein R₅ is C₅-C₁₂ alkyl;
(iv) (2E,6Z)-2,6-nonadienal.

In a particular embodiment, the compound of formula (III) is

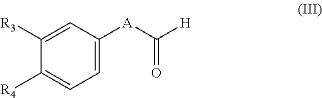

wherein
R₃ is C₁-C₆ alkyl, or C₁-C₆ alkoxy;
R₄ is H, —OH, C₁-C₆ alkyl optionally substituted by a hydroxy group, or C₁-C₆ alkoxy;
A is a divalent radical selected from the group consisting of a bond, C₁-C₆ alkylene, or C₂-C₆ alkenylene.

In a particular embodiment, the compound of formula (III) is

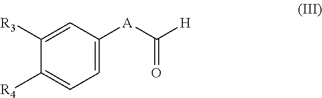

wherein
R₃ is H, C₁-C₆ alkyl, or C₁-C₆ alkoxy;
R₄ is OH, C₁-C₆ alkyl optionally substituted by a hydroxy group, or C₁-C₆ alkoxy;
A is a divalent radical selected from the group consisting of a bond, C₁-C₆ alkylene, or C₂-C₆ alkenylene.

In a particular embodiment, the compound of formula (III) is

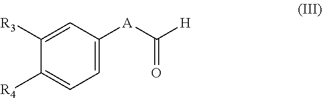

wherein
R₃ is H, C₁-C₆ alkyl, or C₁-C₆ alkoxy;
R₄ is H, OH, C₁-C₆ alkyl optionally substituted by a hydroxy group, or C₁-C₆ alkoxy;
A is a divalent radical selected from the group consisting of a bond, C₂-C₆ alkylene, or C₂-C₆ alkenylene.

In a particular embodiment, the compound of formula (III) is para-anisaldehyde; ethyl vanillin or a compound of formula

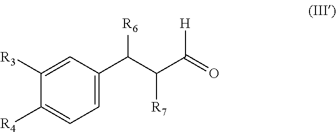

wherein
R₃ is H, C₁-C₆ alkyl, or C₁-C₆ alkoxy;
R₄ is H, —OH, C₁-C₆ alkyl, or C₁-C₆ alkoxy;
R₆ is H, C₁-C₂ alkyl;
R₇ is H, C₁-C₂ alkyl;
   or a compound of formula

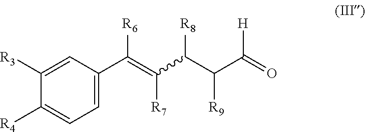

wherein
R₃ is H, C₁-C₆ alkyl, or C₁-C₆ alkoxy;
R₄ is H, OH, C₁-C₆ alkyl, or C₁-C₆ alkoxy;
R₆ is H, C₁-C₂ alkyl;
R₈ is H, C₁-C₂ alkyl;
R₉ is H, C₁-C₂ alkyl.

For the sake of clarity, by the wavy bond in compound of formula (III″), or the similar, it is meant the normal meaning understood by a person skilled in the art, i.e. that the double bond may have a cis configuration corresponding to the Z isomer, a trans configuration corresponding to the E isomer or a mixture thereof. In other words, the compound of formula (III′) may be in the form of its E or Z isomer or of a mixture thereof. In particular, compound (III″) can be in the form of a mixture consisting of isomers E and Z and wherein said isomer E represents at least 25% of the total mixture, at least 35%, at least 50%, or even at least 75% (i.e. a mixture E/Z comprised between 75/25 and 100/0).

In a particular embodiment, in compound of formula (III′) or (III″), R₆ is H or methyl.

In a particular embodiment, in compound of formula (III′) or (III″), R₇ is H or methyl.

In a particular embodiment, in compound of formula (III″), R₈ is H or methyl.

In a particular embodiment, in compound of formula (III″), R₉ is H or methyl.

In a particular embodiment, in compound of formula (III″), at least one, preferably at least two groups among R₆, R₇, R₈ and R₉ are hydrogen and the others are either H or methyl.

In a particular embodiment, the compound of formula (II) is selected from: methyl octine carbonate, cyclopropylmethyl 3-hexenoate, and ethyl 2-methylbutanoate.

In a particular embodiment, the compound of formula (III) is selected from: para-anisyl propanal, cyclamen propanal, (4E)-4-methyl-5-(4-methylphenyl)-4-pentenal, (4E)-2,4-dimethyl-5-(4-methylphenyl)-4-pentenal, cyclamen aldehyde, floral butanal, lilial, ethyl vanillin, 3-[4-(2-hydroxy-2-methylpropyl)phenyl]propanal, and para-anisaldehyde.

In a particular embodiment, the compound of formula (IV) is selected from: 1-decanol, 1-dodecanol, 1-hexanol, and 2-ethyl-1-hexanol.

In a particular embodiment, the composition further comprises one or more of the following compounds: p-menthan-7-ol, gamma-dodecalactone, methyl dihydrojasmonate, cinnamaldehyde, citral, lemongrass, cinnamon oil, delta-undecalactone, litsea cubeba oil, and 10-undecenal.

The compounds used in the anti-dandruff composition are discussed below.

“Methyl octine carbonate” is a material known in the art. It has a floral, green, violet, melon, cucumber odor and a green, melon, cucumber, violet, tropical, fruity flavor. By “methyl octine carbonate”, methyl 2-nonynoate and material registered under CAS 111-80-8 that is available from many sources, is included.

“Cyclopropylmethyl 3-hexenoate” is a material known in the art. Material registered under CAS 1253411-72-1 available from many sources is included.

“Ethyl 2-methylbutanoate” is a material known in the art. It has a sharp, sweet, green apple, fruity odor and a fruity, fresh, berry, grape, pineapple, mango and cherry flavor. By “ethyl 2-methylbutanoate”, (+/−)-ethyl 2-methylbutanoate, ethyl 2-methyl butyrate, and material registered under CAS 7452-79-1 available from many sources is included.

“Para-anisyl propanal” is a material known in the art. It has a sweet, aniseed, basil, fennel, raspberry, green odor. By “para-anisyl propanal”, (+/−)-3-(4-methoxyphenyl)-2-methylpropanal and material registered under CAS 5462-06-6 available from many sources is included.

“Cyclamen propanal” is a material known in the art. It has a fresh, watery, floral, cyclamen, sweet odor. By “cyclamen propanal”, 3-(4-tert-butylphenyl)propanal and material registered under CAS 18127-01-0 available from many sources is included.

“(4E)-4-Methyl-5-(4-methylphenyl)-4-pentenal” is a material known in the art. By “(4E)-4-methyl-5-(4-methylphenyl)-4-pentenal”, material registered under CAS 1226911-69-8 available from many sources is included.

“Cyclamen aldehyde” is a material known in the art. It has a floral, cyclamen, fresh, rhubarb, musty, green odor and a green, tropical, rhubarb, watery, oily, cyclamen, cortex, phenolic flavor. By “cyclamen aldehyde”, (+/−)-3-(4-isopropylphenyl)-2-methylpropanal and material registered under CAS 103-95-7 available from many sources is included.

“Floral butanal” is a material known in the art. It has a floral, green, muguet, fresh, powerful odor. By “floral butanal”, (+/−)-3-(3-isopropylphenyl)butanal and material registered under CAS 125109-85-5 available from many sources is included. Said material is also known as Florhydral in the art.

“Lilial” is a material known in the art. It has a floral, muguet, watery, green, powdery, cumin odor. By “lilial”, (+/−)-2-methyl-3-[4-(2-methyl-2-propanyl)phenyl]propanal and material registered under CAS 80-54-6 available from many sources is included.

“Ethyl vanillin” is a material known in the art. It has a sweet, creamy, vanilla, caramel odor and a sweet, creamy, vanilla, smooth and caramellic flavor. By “ethyl vanillin”, 3-ethoxy-4-hydroxybenzaldehyde and material registered under CAS 121-32-4 available from many sources is included.

“Para-anisaldehyde” is a material known in the art. It has a sweet, powdery, mimosa, floral, hawthorn, balsam odor and a creamy, powdery, vanilla and spicy with a typical marshmallow flavor. By “para-anisaldehyde”, 4-methoxybenzaldehyde and material registered under CAS 123-11-5 available from many sources is included.

“1-Decanol” is a material well known in the art. It has a fatty, waxy, floral, orange, sweet, clean, watery odor and a waxy, green, fatty, floral, costus, watery, citrus flavor. By “1-decanol”, material registered under CAS 112-30-1 that is commercially available from many sources is included.

“1-Dodecanol” is a material well known in the art. It has an earthy, soapy, waxy, fatty, honey, coconut odor and a soapy, waxy, clean, aldehydic, earthy, cilantro, watery, fatty, greasy flavor. By “1-dodecanol”, material registered under CAS 112-53-8 that is commercially available from many sources is included.

“1-Hexanol” is a material well known in the art. It has pungent, etherial, fusel oil, fruity and alcohol odor and a green, fruity, apple-skin and oily flavor. By “1-hexanol”, material registered under CAS 111-27-3 that is commercially available from many sources is included.

“2-Ethyl-1-hexanol” is a material well known in the art. It has citrus, fresh, floral, oily, sweet odor and a sweet, fatty, fruity flavor. By “2-ethyl-1-hexanol”, material registered under CAS 104-76-7 that is commercially available from many sources is included.

“(2E,6Z)-2,6-Nonadienal” is a material well known in the art. It has a green, fatty, dry, cucumber odor and a green, cucumber, melon, fatty and rindy flavor. By “(2E,6Z)-2,6-nonadienal”, material registered under CAS 557-48-2 available from many sources is included.

“(2E)-2-Dodecenal” is a material well known in the art. It has a citrus, metallic, mandarin, orange, waxy, aldehydic odor and a waxy, citrus, peel, aldehydic, bitter, orange, mandarin, cilantro, fatty, herbal flavor. By “(2E)-2-dodecenal”, material registered under CAS 20407-84-5 available from many sources is included.

“Indole” is a material well known in the art. It has an animal, floral, moth, ball, fecal, naphthalene odor and a pungent, floral, slightly naphtha and mothball like with a fecal and animalic musty character flavor. By “indole”, material registered under CAS 120-72-9 available from many sources is included.

“Methyl 2,4-dihydroxy-3,6-dimethylbenzoate” is a material well known in the art. It has a mossy oakmoss woody phenolic earthy odor and a mossy, dry, earthy, woody, phenolic oakmoss, spicy, powdery flavor. By “methyl 2,4-dihydroxy-3,6-dimethylbenzoate”, veramoss, evernyl and material registered under CAS 4707-47-5 available from many sources is included.

“Geranium rose oil” is a material well known in the art. It has a floral, green, spicy, red, rose, dill odor and a floral flavor. By “geranium rose oil”, material registered under CAS 8000-46-2 available from many sources is included.

“4-Methyl-2-pentylpyridine” is a material well known in the art. By “4-methyl-2-pentylpyridine”, material registered under CAS 84625-54-7 available from many sources is included.

“p-Menthan-7-ol” is a material well known in the art. It has a fresh, clean, floral, magnolia, muguet, cumin, grassy odor and a fresh soft and clean floral odor. By “p-menthan-7-ol”, trans-4-(2-propanyl)cyclohexyl]methanol+[cis-4-(2-propanyl)cyclohexyl]methanol, and material registered under CAS 5502-75-0 that is commercially available from many sources, is included.

“Gamma-dodecalactone” is a material well known in the art. It has a fatty, peach, sweet, metallic, fruity odor and a fruity, fatty, creamy, buttery, peach flavor. By “gamma-dodecalactone”, (+/−)-5-octyldihydro-2(3h)-furanone) and material registered under CAS 2305-05-7 that is commercially available from many sources is included.

“Methyl dihydrojasmonate” is a material well known in the art. It has a floral, oily, jasmin, green, lactonic, tropical, natural odor and a sweet, floral, citrus, fruity and berry flavor. By “methyl dihydrojasmonate”, methyl 2-((1 RS,2RS)-3-oxo-2-pentylcyclopentyl)acetate and material registered under CAS 2570-03-8 that is commercially available from many sources is included.

“Cinnamaldehyde” is a material well known in the art. It has a cinnamon spicy cassia odor and a spicy, cinnamon sweet flavor. By “cinnamaldehyde”, (E)-3-phenyl-2-propenal and material registered under CAS 104-55-2 available from many sources is included.

“Citral” is a material well known in the art. It has a sharp, lemon sweet odor and a fresh, juicy, lemon peel flavor. By “citral”, (Z)-3,7-dimethyl-2,6-octadienal+(E)-3,7-dimethyl-2,6-octadienal and material registered under CAS 5392-40-5 that is available from many sources is included.

“Lemongrass oil” is a material well known in the art. It has a fresh, sweet, lemon, grassy, green odor and a green, citrus, lemon, floral, linalool, spice and chamomile-like flavor. By “lemongrass oil”, material registered under CAS 8007-02-1 that is available from many sources, is included.

“Cinnamon oil” is a material well known in the art. It has a sweet, spicy, aromatic, fruity, aldehydic, woody odor and a spicy sweet aromatic aldehydic fruity honey woody flavor. By “cinnamon oil”, cinnamon bark oil and material registered under CAS 8015-91-6 that is available from many sources, is included.

“Delta-undecalactone” is a material well known in the art. It has a creamy, fatty, coconut, fruity odor and a creamy, coconut, milky, creamy, butter fat flavor. By “delta-undecalactone”, 6-hexyltetrahydro-2H-pyran-2-one and material registered under CAS 710-04-3 that is available from many sources, is included.

“Litsea cubeba oil” is a material well known in the art. It has a sweet, fresh, lemon, citral, grassy odor and a citrus, lemon, lemongrass, aldehydic grassy flavor. By “litsea cubeba oil”, material registered under CAS 68855-99-2 that is available from many sources, is included.

“10-Undecenal” is a material well known in the art. It has a waxy, aldehydic, rose, mandarin, citrus, soapy, fatty odor and a fatty, oily and aldehydic flavor. By “10-undecenal”, material registered under CAS 112-45-8 that is available from many sources,is included.

Preferably, the anti-dandruff composition of the invention comprises one or more compounds, which have a positive result in two or more of the assays as described above. In a particular embodiment, the anti-dandruff composition comprises one or more of the following compounds: methyl octine carbonate, cyclopropylmethyl 3-hexenoate, para-anisyl propanal, cyclamen propanal, (4E)-4-methyl-5-(4-methylphenyl)-4-pentenal, cyclamen aldehyde, floral butanal, lilial, ethyl vanillin, para-anisaldehyde, 1-decanol, 1-dodecanol, 2-ethyl-1-hexanol, (2E,6Z)-2,6-nonadienal, (2E)-2-dodecenal, indole, methyl 2,4-dihydroxy-3,6-dimethylbenzoate, geranium rose oil, 4-methyl-2-pentylpyridine, p-menthan-7-ol, gamma-dodecalactone, methyl dihydrojasmonate, cinnamaldehyde, citral, lemongrass, cinnamon oil, delta-undecalactone, litsea cubeba oil, 10-undecenal, (E)-2-nonenal, (E)-2-octenal, (4-methylphenyl)acetaldehyde, (2E)-2-methyl-3-phenyl-2-propenal, 2,4-dimethyl-3-cyclohexene-1-carbaldehyde, 2,4-dimethyl-3-cyclohexene-1-carbaldehyde, 3-(4-ethylphenyl)-2,2-dimethylpropanal, 2,6-dimethyl-2-heptanol, 3-methyl-5-phenylpentanal, octanal, 3-(3-isopropyl-1-phenyl)butanal, nonanal, (Z)-6-nonenal, 2-phenylpropanal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~.0~4,6~]dodecane, (1RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1RS)-1-[(1SR)-3,3-dimethylcyclohexyl]ethoxy}-2-oxoethyl propionate, 2-{(1 RS)-1-[(1 RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2H-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1 r)-2,2,3-trimethyl-3-cyclopenten-1-yl]-4-pentenal.

In a particular embodiment, the anti-dandruff composition comprises one or more of the following compounds: methyl octine carbonate, cyclopropylmethyl 3-hexenoate, para-anisyl propanal, cyclamen propanal, (4E)-4-methyl-5-(4-methylphenyl)-4-pentenal, cyclamen aldehyde, floral butanal, lilial, ethyl vanillin, para-anisaldehyde, 1-decanol, 1-dodecanol, 2-ethyl-1-hexanol, (2E,6Z)-2,6-nonadienal, (2E)-2-dodecenal, indole, methyl 2,4-dihydroxy-3,6-dimethylbenzoate, geranium rose oil, 4-methyl-2-pentylpyridine, p-menthan-7-ol, gamma-dodecalactone, methyl dihydrojasmonate, cinnamaldehyde, citral, lemongrass, cinnamon oil, delta-undecalactone, litsea cubeba oil, and 10-undecenal.

Preferably, the anti-dandruff composition comprises one or more of the following compounds: p-menthan-7-ol, (4E)-4-methyl-5-(4-methylphenyl)-4-pentenal, 4-methyl-2-pentylpyridine, methyl octine carbonate, gamma-dodecalactone, methyl 2,4-dihydroxy-3,6-dimethylbenzoate, (2E)-2-dodecenal, 2-ethyl-1-hexanol, cyclopropylmethyl 3-hexenoate, cyclamen aldehyde, citral, (2E,6Z)-2,6-nonadienal, cinnamon oil, 1-dodecanol, cyclamen propanal, 10-undecenal, para-anisyl propanal, 1-decanol, geranium rose oil, litsea cubeba oil, delta-undecalactone.

More preferably, the anti-dandruff composition comprises one or more of the following compounds: p-menthan-7-ol, 4-methyl-2-pentylpyridine, methyl 2,4-dihydroxy-3,6-dimethylbenzoate, cyclopropylmethyl 3-hexenoate, citral, (2E,6Z)-2,6-nonadienal, 1-dodecanol, cyclamen propanal, 10-undecenal, 1-decanol, geranium rose oil, litsea cubeba oil, delta-undecalactone.

In a particular embodiment, the anti-dandruff composition of the invention comprises one or more compounds, which have a positive result in all four of the assays described above, i.e. which are effective against all four bacteria as mentioned above. Hence, in a particular preferred embodiment, the composition comprises one or more of the following compounds: (2E,6Z)-2,6-nonadienal, (2E)-2-dodecenal, 1-decanol, cyclopropylmethyl 3-hexenoate, para-anisyl propanal, (4E)-4-methyl-5-(4-methylphenyl)-4-pentenal, 2-ethyl-1-hexanol, methyl 2,4-dihydroxy-3,6-dimethylbenzoate, geranium rose oil, 4-methyl-2-pentylpyridine, cinnamaldehyde, cinnamon oil, citral, delta-undecalactone, litsea cubeba oil, and p-menthan-7-ol.

As stated above, the anti-dandruff composition of the invention comprises one or more of the compounds or compositions described herein. In a particular embodiment, the composition of the invention comprises two, three, four, five, six, seven, eight, nine, ten, eleven, twelve, thirteen, fourteen, fifteen, sixteen, seventeen, eighteen, nineteen, twenty, 21, 22, 23, 24, 25, 26, 27 28, 29, 30, or more of the following compounds: methyl octine carbonate, cyclopropylmethyl 3-hexenoate, para-anisyl propanal, cyclamen propanal, (4E)-4-methyl-5-(4-methylphenyl)-4-pentenal, cyclamen aldehyde, floral, butanal, lilial, ethyl vanillin, para-anisaldehyde, 1-decanol, 1-dodecanol, 2-ethyl-1-hexanol, (2E,6Z)-2,6-nonadienal, (2E)-2-dodecenal, indole, methyl 2,4-dihydroxy-3,6-dimethylbenzoate, geranium rose oil, 4-methyl-2-pentylpyridine, p-menthan-7-ol, gamma-dodecalactone, methyl dihydrojasmonate, cinnamaldehyde, citral, lemongrass, cinnamon oil, delta-undecalactone, litsea cubeba oil, 10-undecenal, ethyl 2-methyl butanoate, and 1-hexanol.

In a particular embodiment, the anti-dandruff composition according to the present invention is an anti-dandruff composition (ADC-1) comprising the following compounds: (2E,6Z)-2,6-nonadienal, (E)-3-phenyl-2-propenal, cinnamon oil ceylon, and 2-methoxy-4-(2-propen-1-yl)phenol.

Preferably, the total amount of said compounds in ADC-1 is from 0.1-0.5 wt. %, more preferably is 0.2 wt. %. Preferably each of the compounds individually is present in an amount of from 0.01-1 wt. %.

In a particular embodiment, the anti-dandruff composition according to the present invention is an anti-dandruff composition (ADC-2) comprising the following compounds: (2E,6Z)-2,6-nonadienal, (E)-3-phenyl-2-propenal, (+/−)-3-(4-methoxyphenyl)-2-methylpropanal, (2Z)-3,7-dimethyl-2,6-octadienal, and (2E)-3,7-dimethyl-2,6-octadienal.

Preferably, the total amount of said compounds in ADC-2 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-3) comprising the following compounds: (2E,6Z)-2,6-nonadienal, (E)-3-phenyl-2-propenal, (2E)-2-dodecenal, (2Z)-3,7-dimethyl-2,6-octadienal, and (2E)-3,7-dimethyl-2,6-octadienal.

Preferably, the total amount of said compounds in ADC-3 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-4) comprising the following compounds: (2E,6Z)-2,6-nonadienal, (E)-3-phenyl-2-propenal, (4E)-4-methyl-5-(4-methylphenyl)-4-pentenal, (2Z)-3,7-dimethyl-2,6-octadienal, and (2E)-3,7-dimethyl-2,6-octadienal.

Preferably, the total amount of said compounds in ADC-4 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-5) comprising the following compounds: (2E)-2-dodecenal, (+/−)-3-(4-methoxyphenyl)-2-methylpropanal, (4E)-4-methyl-5-(4-methylphenyl)-4-pentenal, and octanoic acid.

Preferably, the total amount of said compounds in ADC-5 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-6) comprising the following compounds: (E)-3-phenyl-2-propenal, (2E)-2-dodecenal, (+/−)-3-(4-methoxyphenyl)-2-methylpropanal, and octanoic acid.

Preferably, the total amount of said compounds in ADC-6 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-7) comprising the following compounds: cinnamon oil ceylon, (2E)-2-dodecenal, (+/−)-3-(4-methoxyphenyl)-2-methylpropanal, and octanoic acid.

Preferably, the total amount of said compounds in ADC-7 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-8) comprising the following compounds: (2E,6Z)-2,6-nonadienal, (2E)-2-dodecenal, (4E)-4-methyl-5-(4-methylphenyl)-4-pentenal, and octanoic acid.

Preferably, the total amount of said compounds in ADC-8 is from 0.1-2 wt. %, more preferably is 0.5 wt. %. Preferably each of the compounds individually is present in an amount of from 0.05-0.55 wt. %.

In a particular embodiment, the anti-dandruff composition according to the present invention is an anti-dandruff composition (ADC-9) comprising the following compounds: (2E,6Z)-2,6-nonadienal, (2E)-2-dodecenal, nonanoic acid, and decanoic acid.

Preferably, the total amount of said compounds in ADC-9 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-10) comprising the following compounds: (2E)-2-dodecenal, (4E)-4-methyl-5-(4-methylphenyl)-4-pentenal, nonanoic acid, (2Z)-3,7-dimethyl-2,6-octadienal, and (2E)-3,7-dimethyl-2,6-octadienal.

Preferably, the total amount of said compounds in ADC-10 is from 0.1-2 wt. %, more preferably is 1 wt. %. Preferably each of the compounds individually is present in an amount of from 0.05-0.55 wt. %.

In a particular embodiment, the anti-dandruff composition according to the present invention is an anti-dandruff composition (ADC-11) comprising the following compounds: (2E,6Z)-2,6-nonadienal and (E)-3-phenyl-2-propenal.

Preferably, the total amount of said compounds in ADC-11 is from 0.1-1 wt. %, more preferably is 0.2 wt. %. Preferably each of the compounds individually is present in an amount of from 0.01-0.15 wt. %.

In a particular embodiment, the anti-dandruff composition according to the present invention is an anti-dandruff composition (ADC-12) comprising the following compounds: (E)-3-phenyl-2-propenal, (2E)-2-dodecenal, and (+/−)-3-(4-methoxyphenyl)-2-methylpropanal.

Preferably, the total amount of said compounds in ADC-12 is from 0.1-1 wt. %, more preferably is 0.5 wt. %. Preferably each of the compounds individually is present in an amount of from 0.1-0.2 wt. %.

In a particular embodiment, the anti-dandruff composition according to the present invention is an anti-dandruff composition (ADC-13) comprising the following compounds: (E)-3-phenyl-2-propenal, (2E)-2-dodecenal, and (4E)-4-methyl-5-(4-methylphenyl)-4-pentenal.

Preferably, the total amount of said compounds in ADC-13 is from 0.1-1 wt. %, more preferably is 0.5 wt. %. Preferably each of the compounds individually is present in an amount of from 0.1-0.2 wt. %.

In a particular embodiment, the anti-dandruff composition according to the present invention is an anti-dandruff composition (ADC-14) comprising the following compounds: (E)-3-phenyl-2-propenal, (+/−)-3-(4-methoxyphenyl)-2-methylpropanal, and (4E)-4-methyl-5-(4-methylphenyl)-4-pentenal.

Preferably, the total amount of said compounds in ADC-14 is from 0.1-1 wt. %, more preferably is 0.5 wt. %. Preferably each of the compounds individually is present in an amount of from 0.1-0.2 wt. %.

In a particular embodiment, the compound of formula (I) is any of nonanoic acid, octanoic acid, decanoic acid, (2E,4E)-2,4-heptadienal, (E,E)-2,6-nonadienal, (E)-2-nonen-1-ol, (3S)-3,7-dimethyl-6-octen-1-ol, (Z)-5-octen-1-ol, (Z)-3-octen-1-ol, (E)-2-octenal, (4-methylphenyl)acetaldehyde, (2E)-2-methyl-3-phenyl-2-propenal, 2,4-dimethyl-3-cyclohexene-1-carbaldehyde, 2,4-dimethyl-3-cyclohexene-1-carbaldehyde, 3-(4-ethylphenyl)-2,2-dimethylpropanal, 2,6-dimethyl-2-heptanol, 3-methyl-5-phenylpentanal, octanal, 3-(3-isopropyl-1-phenyl)butanal, nonanal, (Z)-6-nonenal, 2-phenylpropanal, and any mixture thereof.

In a particular embodiment, the compound in the anti-dandruff composition is one or more of the following perfume compounds: (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1 RS,2RS,4RS,6SR,8RS,9RS,11E)-11-ethylidene-4-methyl-5-oxatetracyclo[7.2.1.0~2,8~.0~4,6~]dodecane, (1RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1 RS,2RS,7SR,8RS,9Z)-9-ethylidene-3-oxatricyclo[6.2.1.0~2,7~]undecane, (1RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1SR)-3,3-dimethylcyclohexyl]ethoxy}-2-oxoethyl propionate, 2-{(1 RS)-1-[(1 RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2H-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal. In a particular embodiment, the anti-dandruff composition comprises one or more of the following compounds: (E)-2-nonenal, (E)-2-octenal, (4-methylphenyl)acetaldehyde, (2E)-2-methyl-3-phenyl-2-propenal, 2,4-dimethyl-3-cyclohexene-1-carbaldehyde, 2,4-dimethyl-3-cyclohexene-1-carbaldehyde, 3-(4-ethylphenyl)-2,2-dimethylpropanal, 2,6-dimethyl-2-heptanol, 3-methyl-5-phenylpentanal, octanal, 3-(3-isopropyl-1-phenyl)butanal, nonanal, (Z)-6-nonenal, 2-phenylpropanal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1 RS,2RS,4RS,6SR,8RS,9RS,11E)-11-ethylidene-4-methyl-5-oxatetracyclo[7.2.1.0~2,8~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1 RS,2RS,7SR,8RS,9Z)-9-ethylidene-3-oxatricyclo[6.2.1.0~2,7~]undecane, (1RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1SR)-3,3-dimethylcyclohexyl]ethoxy}-2-oxoethyl propionate, 2-{(1 RS)-1-[(1 RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2H-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, 3-[4-(2-hydroxy-2-methylpropyl)phenyl]propanal, and (2S)-2-methyl-4-[(1R)-2,2,3-trimethyl-3-cyclopenten-1-yl]-4-pentenal.

As can be appreciated, the amount of the compounds in the composition can vary based on the specific compounds used and the intended use. In a particular embodiment, the compounds are present in the anti-dandruff composition in an effective amount.

The term “effective amount” refers to an amount necessary to obtain the desired physiological effect. The physiological effect may be achieved by one application dose or by repeated applications. The dosage administered may, of course, vary depending upon known factors, such as the physiological characteristics of the particular composition and its mode and route of administration; the age and health of the recipient; the kind of concurrent treatment; the frequency of treatment; and the effect desired and can be adjusted by a person skilled in the art. In particular, the term “effective amount” includes within its meaning a non or low toxic but sufficient amount of the compounds to provide the desired effect (i.e. improvement in dandruff). In particular, the amount would be sufficient to reduce the growth of Malassezia furfur, Malassezia globosa and/or Malassezia restricta, but low enough to avoid serious side effects (e.g. undue toxicity or allergic reaction).

In a particular embodiment, the compounds are present at a concentration of at least 0.01 wt. %, 0.05 wt. %, 0.1 wt. %, 0.5 wt. %, 1 wt. %, 2 wt. %, or more, based on the total weight of the anti-dandruff composition.

In a particular embodiment, the compounds are present at a maximum concentration of 100 wt. %, 95 wt. %, 90 wt. %, 85 wt. %, or less, based on the total weight of the anti-dandruff composition.

In a particular embodiment, the concentration range is from 0.5 wt. % to 90 wt. %, preferably from 1 wt. % to 80 wt. %, with respect to the total weight of the anti-dandruff composition.

In a preferred embodiment, the compounds are present at a concentration of at least 5 wt. %, more preferably at least 10 wt. %, most preferably at least 20 wt. %, based on the total weight of the anti-dandruff composition.

As shown in the accompanying examples, the inventors prepared a series of compositions, which comprised combinations of the compounds described herein. The series compositions showed good use in the reduction of one or more of Malassezia furfur, Malassezia globosa and Malassezia restricta growth.

Thus, in another preferred embodiment, the anti-dandruff composition of the invention are the following anti-dandruff compositions:
1. ADC-15 comprising: 1-dodecanol and 1-decanol. Preferably, the composition comprises from 20-80 wt. % 1-dodecanol and from 20-80 wt. % 1-decanol. More preferably, the composition comprises from 40-60 wt. % 1-dodecanol and from 40-60 wt. % 1-decanol. Most preferably, the composition comprises 55.5 wt. % 1-dodecanol and 40.0 wt. % 1-decanol. This is termed herein “mix 1”.
2. ADC-16 comprising: 1-dodecanol and p-menthan-7-ol. Preferably, the composition comprises from 20-80 wt. % 1-dodecanol and from 20-80 wt. % p-menthan-7-ol. More preferably, the composition comprises from 50-80 wt. % 1-dodecanol and from 10-30 wt. % p-menthan-7-ol. Most preferably, the composition comprises 75.2 wt. % 1-dodecanol and 20.1 wt. % p-menthan-7-ol. This is termed herein “mix 2”.
3. ADC-17 comprising: 1-decanol, p-menthan-7-ol, gamma-dodecalactone, methyl dihydrojasmonate and geranium rose oil. Preferably, the composition comprises: 10-30 wt. % 1-decanol, 10-50 wt. % p-menthan-7-ol, 5-20 wt. % gamma-dodecalactone, 10-50 wt. % methyl dihydrojasmonate, and 1-10 wt. % geranium rose oil. More preferably, the composition comprises: 10-20 wt. % 1-decanol, 15-35 wt. % p-menthan-7-ol, 5-20 wt. % gamma-dodecalactone, 15-35 wt. % methyl dihydrojasmonate, and 2-7 wt. % geranium rose oil. Most preferably, the composition comprises: 17.8 wt. % 1-decanol, 30 wt. % p-menthan-7-ol, 10 wt. % gamma-dodecalactone, 30 wt. % methyl dihydrojasmonate, and 5 wt. % geranium rose oil. This is termed herein “mix 3”.
4. ADC-18 comprising: 1-dodecanol, 1-decanol, cinnamaldehyde, and citral. Preferably, the composition comprises: 20-80 wt. % 1-dodecanol, 10-30 wt. % 1-decanol, 1-10 wt. % cinnamaldehyde, and 15-30 wt. % citral. More preferably, the composition comprises: 45-55 wt. % 1-dodecanol, 15-30 wt. % 1-decanol, 2-7 wt. % cinnamaldehyde, and 15-25 wt. % citral. Most preferably, the composition comprises: 53.7 wt. % 1-dodecanol, 20 wt. % 1-decanol, 5 wt. % cinnamaldehyde, and 20 wt. % citral. This is termed herein “mix 4”.
5. ADC-19 comprising: cinnamaldehyde, citral, 10-undecenal, p-menthan-7-ol, and cyclopropylmethyl 3-hexenoate. Preferably, the composition comprises: 10-40 wt. % cinnamaldehyde, 20-60 wt. % citral, 2 wt. % 10-undecenal, 5-40 wt. % p-menthan-7-ol, and 1-10 wt. % cyclopropylmethyl 3-hexenoate. More preferably, the composition comprises: 25-35 wt. % cinnamaldehyde, 30-45 wt. % citral, 1-5 wt. % 10-undecenal, 20-25 wt. % p-menthan-7-ol, and 2-7 wt. % cyclopropylmethyl 3-hexenoate. Most preferably, the composition comprises: 30 wt. % cinnamaldehyde, 40 wt. % citral, 2 wt. % 10-undecenal, 23 wt. % p-menthan-7-ol, and 5 wt. % cyclopropylmethyl 3-hexenoate. This is termed herein “mix 5”.
6. ADC-20 comprising: cinnamaldehyde, citral, 10-undecenal, p-menthan-7-ol, and cyclopropylmethyl 3-hexenoate. Preferably, the composition comprises: 20-60 wt. % cinnamaldehyde, 1-20 wt. % citral, 1-15 wt. % 10-undecenal, 20-60 wt. % p-menthan-7-ol, and 1-20 wt. % cyclopropylmethyl 3-hexenoate. More preferably, the composition comprises: 35-45 wt. % cinnamaldehyde, 7-15 wt. % citral, 2-6 wt. % 10-undecenal, 35-40 wt. % p-menthan-7-ol, and 2-5 wt. % cyclopropylmethyl 3-hexenoate. Most preferably, the composition comprises: 44 wt. % cinnamaldehyde, 11 wt. % citral, 4 wt. % 10-undecenal, 38 wt. % p-menthan-7-ol, and 3 wt. % cyclopropylmethyl 3-hexenoate. This is termed herein “mix 6”.
7. ADC-21 comprising: lemongrass, cinnamon oil, and cyclopropylmethyl 3-hexenoate. Preferably, the composition comprises: 1-20 wt. % lemongrass, 20-70 wt. % cinnamon oil, and 20-60 wt. % cyclopropylmethyl 3-hexenoate. More preferably, the composition comprises: 5-15 wt. % lemongrass, 40-60 wt. % cinnamon oil, and 35-45 wt. % cyclopropylmethyl 3-hexenoate. Most preferably, the composition comprises: 10 wt. % lemongrass, 50 wt. % cinnamon oil, and 40 wt. % cyclopropylmethyl 3-hexenoate. This is termed herein “mix 7”.
8. ADC-22 comprising: (2E,6Z)-2,6-nonadienal, cinnamon oil, delta-undecalactone, and litsea cubeba oil. Preferably, the composition comprises: 10-30 wt. % (2E,6Z)-2,6-nonadienal, 5-20 wt. % cinnamon oil, 10-30 wt. % delta-undecalactone, and 30-70 wt. % litsea cubeba oil. More preferably, the composition comprises: 10-20 wt. % (2E,6Z)-2,6-nonadienal, 7-12 wt. % cinnamon oil, 20-25 wt. % delta-undecalactone, and 45-55 wt. % litsea cubeba oil. Most preferably, the composition comprises: 18.5 wt. % (2E,6Z)-2,6-nonadienal, 10 wt. % cinnamon oil, 21.5 wt. % delta-undecalactone, and 50 wt. % litsea cubeba oil. This is termed herein “mix 8”.

In addition, the inventors prepared a further composition which combines a number of compounds as described herein. This also shows showed good use in the reduction of one or more of Malassezia furfur, Malassezia globosa and Malassezia restricta growth. The compounds used in this mixture have previously been shown to have antimicrobial activity. However, this is the first time a combination of these compounds and compositions has been prepared and shown to retain efficacy.

Accordingly, in a preferred embodiment the anti-dandruff composition is an anti-dandruff composition (ADC-23) comprising: citral, delta-undecalactone and p-menthan-7-ol. Preferably, the composition comprises 20-80 wt. % citral, 10-40 wt. % delta-undecalactone and 5-30 wt. % p-menthan-7-ol. More preferably, the composition comprises 55-65 wt. % citral, 25-35 wt. % delta-undecalactone and 7-12 wt. % p-menthan-7-ol. Most preferably, the composition comprises 60 wt. % citral, 30 wt. % delta-undecalactone and 10 wt. % p-menthan-7-ol. This is termed herein “mix 9”.

In a particular embodiment, hair care products comprising the anti-dandruff compositions according to the present invention such as the anti-dandruff compositions ADC-1, ADC-2, ADC-3, ADC-4, ADC-5, ADC-6, ADC-7, ADC-8, ADC-9, ADC-10, ADC-11, ADC-12, ADC-13, ADC-14, ADC-15, ADC-16, ADC-17, ADC-18, ADC-19, ADC-20, ADC-21, ADC-22 and ADC-23 further comprise one or more anti-dandruff agents. Such agents are well known in the art. Examples include: olamine, hexamidine diisethionate, dichlorobenzyl alcohol (DCBA), and selenium sulfide, nystatin, cuprimyxin, tolnaftate, candicidin, haloprogin, iodochlorohydroxyquin, clotrimazole, undecylenic acid, proprionic acid, caprylic acid, benzoic acid, salicylic acid, griseofulvin, amphotericin B, ketoconazole, miconazole, filipin, hamycin, natamycin, rimocidin, bifonazole, butoconazole, econazole, fenticonazole, isoconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, ciclopirox olamine, flucytosine, crystal violet, piroctone olamine, zinc pyrithione, selenium sulphide, tar, and tea tree oil. Preferably, the other anti-dandruff agents (apart from the perfume compounds having anti-dandruff activity) in the hair care product according to the present invention are selected from the group consisting of hexamidine diisethionate, dichlorobenzyl alcohol (DCBA), piroctone olamine, climbazole, selenium sulphide, coal tar, azelaic acid, ketoconazole, salicylic acid, undecylenic acid, and mixtures thereof.

Typical use levels of piroctone olamine in hair care products according to the present invention are selected in the range of up to 0.01 to 1 wt.-%, preferably in the range of 0.1 to 1 wt.-%.

Typical use levels of salicylic acid in hair care products according to the present invention are selected in the range of up to 0.01 to 2 wt.-%, preferably in the range of 0.1 to 2 wt.-%, most preferably 0.1 to 1 wt.-%.

Typical use levels of ketoconazole in hair care products according to the present invention are selected in the range of up to 0.01 to 2 wt.-%, preferably in the range of 0.1 to 2 wt.-%, most preferably 0.5 to 2 wt.-%.

The present inventors investigated whether the anti-dandruff effect could still be improved when the perfume compounds described above are mixed with common anti-dandruff agents. As shown in the examples, combinations of (2E,6Z)-2,6-nonadienal with piroctone olamine, salicylic acid, and ketoconazole, respectively, lead to increased antimicrobial effects as compared to the use of the individual compounds alone.

Thus, in a particular preferred embodiment, the anti-dandruff composition is an anti-dandruff composition ADC-24 comprising (i) (2E,6Z)-2,6-nonadienal, and (ii) piroctone olamine, salicylic acid, and/or ketoconazole.

Particularly preferred embodiments are the anti-dandruff compositions
ADC-25 comprising piroctone olamine and (2E,6Z)-2,6-nonadienal, preferably in amounts of from 0.1-1% piroctone olamine and 0.1-1% (2E,6Z)-2,6-nonadienal, more preferably in amounts of from 0.19% piroctone olamine and 0.5% or 0.75% (2E,6Z)-2,6-nonadienal;
ADC-26 comprising salicylic acid and (2E,6Z)-2,6-nonadienal, preferably in amounts of from 0.1-1% salicylic acid and 0.5-2% (2E,6Z)-2,6-nonadienal, more preferably in amounts of from 0.5% salicylic acid and 1% (2E,6Z)-2,6-nonadienal; or
ADC-27 comprising ketoconazole and (2E,6Z)-2,6-nonadienal, preferably in amounts of from 0.1%-2% ketoconazole and 0.1-2% (2E,6Z)-2,6-nonadienal, more preferably in amounts of from 0.1%-0.4% ketoconazole and 1% (2E,6Z)-2,6-nonadienal.

The anti-dandruff composition of the present invention can be in the form of elixirs, suspensions, emulsions, solutions, syrups, aerosols (as a solid or in a liquid medium), ointments, or lotions. Liquid forms of the compositions include aqueous solutions, aqueous or oil suspensions, and emulsions.

A further aspect of the invention provides a hair care product comprising the anti-dandruff composition according to the invention. Examples of such hair care products include shampoo, conditioner 1-in-1 shampoo/conditioner, hair coloring product, leave-on hair tonic, hair sunscreen product, styling mousse or gel, sprays, lotions, shower gels or other hair treatment composition.

As can be seen in the accompanying examples, the present inventors prepared several hair care products comprising the anti-dandruff composition according to the invention. They showed that the amount of anti-dandruff composition can be 0.05 wt. % or more with respect to the total amount of the hair care product. A preferred amount of the anti-dandruff composition is 0.075 wt. %, 0.1 wt. %, 0.15 wt. %, 0.2 wt. %, 0.3 wt. %, 0.5 wt. % 1 wt. %, 1.5 wt. %, 2 wt. % or more with respect to the total amount of the hair care product. Preferably, the amount of the anti-dandruff composition according to the invention in the hair care product is in the range of from 0.05 to 5 wt. %, more preferably in the range of from 0.05 to 3 wt. %,even more preferably in the range of from 0.05 to 2 wt. %, most preferably in the range of from 0.1 to 2 wt. % or from 0.05 to 0.1 wt. %.

Preferably, in all embodiments of the present invention, the hair care product comprises at least 0.2 wt %, with respect to the total amount of the hair care product, of one or more compound having an inhibition zone diameter on Malassezia restricta of at least 15 mm; 20 mm, 30 mm, 40 mm or more. Preferably, the hair care product comprises at least 0.5 wt %, with respect to the total amount of the hair care product, of one or more compound having an inhibition zone diameter on Malassezia restricta of at least 15 mm; 20 mm, 30 mm, 40 mm or more. Even more preferably, the hair care product comprises at least 1 wt %, with respect to the total amount of the hair care product, of one or more compound having an inhibition zone diameter on Malassezia restricta of at least 15 mm; 20 mm, 30 mm, 40 mm or more. Optionally, the hair care product comprises at most 2 wt %, with respect to the total amount of the hair care product, of one or more compound having an inhibition zone diameter on Malassezia restricta of at most 15 mm.

The hair care product can comprise surfactants. Hair care products according to the present invention preferably comprise at least one surfactant. The surfactant may be selected from anionic, non-ionic, zwitterionic, amphoteric and/or cationic surfactants. Preferably, the surfactant may be anionic surfactant.

Suitable anionic surfactants include alkyl sulfates, alkyl ether sulfates, alpha olefin sulfonates, sulfosuccinates, isethionates, acyl amides, acyl glutamates, alkyl ether carboxylates and alkyl phosphates. Alkyl ether sulfates and/or alkyl sulfates are preferred, particularly alkali metal, e.g. sodium, and/or ammonium salts thereof. Lauryl ether sulfate and/or lauryl sulfate are particularly preferred anionic surfactants. If present, the concentration of anionic surfactant may preferably be in the range from 0.1 wt. % to 45 wt. %, particularly 0.5 wt. % to 45 wt. %, particularly 1.5 wt. % to 35 wt. %, particularly 5 wt. % to 25 wt. %, even more particularly 7 wt. % to 15 wt. %, based on the total weight of the hair care product.

Suitable non-ionic surfactants include the fatty alcohol acid or amide ethoxylates, alkanolamides and alkoxylated alkanolamides, monoglyceride ethoxylates, sorbitan ester ethoxylates, alkyl polyglycosides, ethylene glycol monoesters, ethylene glycol diesters, and mixtures thereof. If present, the concentration of non-ionic surfactant may preferably be in the range from 0.1 wt. % to 30 wt. %, more preferably 0.5 wt. % to 10 wt. %, particularly 1 wt. % to 5 wt. %, and especially 1.5 wt. % to 2 wt. %, based on the total weight of the hair care product.

Suitable amphoteric surfactants inclu 52 lkylaminoino-diproprionates, alkylamphoglycinates, alkylamphoproprionates, alkylamphoacetates (mono- and di-),N-alkyl beta-aminoproprionic acids, alkylpolyamino carboxylates, phosphorylated imidazolines, and mixtures thereof. If present, the concentration of amphoteric surfactant may preferably be in the range from 0.1 wt. % to 20 wt. %, more preferably 0.5 wt. % to 10 wt. %, particularly 1 wt. % to 5 wt. %, and especially 1.5 wt. % to 2 wt. %, based on the total weight of the hair care product.

Suitable cationic surfactants include alkyl quaternaries, benzyl quaternaries, ester quaternaries, ethoxylated quaternaries, alkyl amines, and mixtures thereof.

The cationic surfactant may also be a polyquaternium material (or polyquat). Polyquats include polymers based on acrylamide and/or dimethyl allylamonium chloride such as Polyquaternium 6, Polyquaternium 7, and the like. Polymeric quaternium ammonium salts of guar gum, such as guar hydroxypropyltrimonium chloride, may be used. Polymeric quaternium ammonium salts of cellulose such as Polyquaternium 10 and the like, and polymeric quaternium ammonium salts of starch, may also be used.

If present, the concentration of cationic surfactant may preferably be in the range from 0.1 wt. % to 20 wt. %, more preferably 0.1 wt. % to 10 wt. %, particularly 0.3 wt. % to 3 wt. %, and especially 0.5 wt. % to 1 wt. % by weight based on the total weight of the hair care product.

If present, the concentration of zwitterionic surfactant may preferably be in the range from 5 to 35 wt.-%, preferably from 10 to 35 wt.-%, based on the total weight of the hair care product. Further suitable ranges are from 7.5 to 25 wt.-%, from 10 to 25 wt.-%, from 7.5 to 10 20 wt.-%, from 10 to 20 wt.-%, from 15-35 wt.-%, from 20-30 wt.-% and from 22-27 wt.-%

In a particular embodiment, the hair care product comprises at least one anionic and/or at least one zwitterionic surfactant, such as in particular a laureth sulfate and/or an alkyl amidopropyl betaine. Preferably, the hair care product comprises as surfactant at least sodium laureth sulfate and/or cocamidopropyl betaine, preferably both, even more preferably as sole surfactants.

In a particular embodiment, the hair care product comprises a total amount of anionic and zwitterionic surfactants from 30 to 75 wt.-%, preferably from 40 to 60 wt.-%. Preferably, the hair care product comprises at least 40 wt.-% of water, more preferably at least 45 wt.-% of water, based on the total weight of the hair care product.

In a particular embodiment, the total amount of surfactants in the hair care product is from 1 to 50 wt.-%, preferably from 2 to 40 wt.-%, more preferably from 5 to 25 wt.-%, such as in particular in the range of from 9 to 15 wt.-%, based on the total weight of the hair care product.

The hair care product according to the invention can be used in a method of treating the scalp, said method comprising the steps of (i) contacting the scalp with a hair care product, preferably shampoo, according to the invention, followed by (ii) rinsing off the shampoo with water, and optionally (Ill) drying the hair with a hair dryer.

The anti-dandruff composition respectively the hair care product can also include other cosmetically acceptable ingredients, and in the case of the hair care product in particular those which are suitable for topical application to the hair.

The anti-dandruff actives can be mixed with or diluted by an excipient in the anti-dandruff composition and/or the hair care product. When the excipient serves as a diluent, it can be a solid, semisolid, or liquid material, which acts as a vehicle, carrier or medium for the active ingredient. Examples of suitable excipients include: lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose.

The anti-dandruff composition and/or the hair care product may additionally comprise: lubricating agents such as talc, magnesium stearate, and mineral oil; wetting agents; emulsifying and suspending agents; preserving agents such as methyl- and propylhydroxy-benzoates; sweetening agents; and flavouring agents.

The anti-dandruff composition and/or the hair care product may comprise water. The amount of water in the anti-dandruff composition and/or the hair care product may suitably be in the range from 10 wt. % to 97 wt. %, preferably 30 wt. % to 95 wt. %, more preferably 50 wt. % to 90 wt. %, particularly 65 wt. % to 85 wt. %, and especially 72 wt. % to 78 wt. %, based on the total weight of the composition or product.

The anti-dandruff composition of the present invention may be used with one or more of the other standard ingredients or carriers used in hair care products, including shine enhancers, moisturisers, herbal additives, hair strengtheners, vitamin additives, colorants, hair thickening agents; setting and styling agents; ultraviolet absorbers; silicone oils; essential oils and fragrances; thickening or viscosity-enhancing agents; detergents; stabilising agents; emollients; chelating agents; sequestering agents; preservatives; disinfectants; anti-oxidants/radical scavengers; antistatic agents; conditioning agents; detangling ingredients; emulsifying or dispersing agents; stimulants; soothers; solvents; carriers and the like. The anti-dandruff composition may be admixed with all or some of said ingredients before being incorporated into the hair care product and/or be combined in the hair care product, however being added separately.

One preferred hair care product according to the present invention is a hair shampoo or conditioner, which functions to make the hair more shiny and manageable. The shampoo or conditioner may be in the form of a dispersion, emulsion or solution. One preferred system is one that forms liquid crystals. The liquid crystals are preferably lyotropic liquid crystals (i.e. both concentration and temperature dependent), more preferably lamellar phase liquid crystals, and particularly L alpha phase (neat) liquid crystals. The concentration of the anti-dandruff composition in the shampoo or conditioner is preferably in the range from 0.1 wt. % to 10 wt. %, more preferably 0.3 wt. % to 2 wt. %, particularly 0.4 wt. % to 1.5 wt. %, and especially 0.5% to 1 wt. % based on the total weight of the shampoo or conditioner composition.

The shampoo or conditioner may contain many different types of functional ingredients such as; cationic hair conditioning agents; fatty alcohols, e.g. stearyl, cetearyl, cetyl, oleyl alcohols, used typically at a concentration range of 2 wt. % to 5 wt. % based on the total weight of the composition; humectants or solvents, e.g. alcohols and polyols such as glycerol and polyethylene glycols, when used typically at a concentration in the range from 1 wt. % to 10 wt. % based on the total weight of the composition; reconstructors, e.g. hydrolysed proteins such as wheat protein, which function to penetrate the hair and strengthen the hair structure through polymer crosslinking; glossing or detangling materials which bind to the hair and reflect light, e.g. silicones such as dimethicone, phenyltrimethicone, dimethiconol and/or trimethylsilylamodimethicone, usually at a concentration in the range from 0.2 wt. % to 10 wt. % based on the total weight of the composition; thermal protectors, usually heat-absorbing polymers, which shield the hair against excessive heat; UV protection agents, to protect hair or formulation components from degradation by UV light.

The hair care product may also be a scalp care product, such as a scalp lotion, serum, oil, scrub, mousse, liquid, cleanser or similar such products. In addition to the standard ingredients or carriers used in hair care products listed above, scalp care products can also contain a scalp conditioning agent which is suitable for conditioning skin that can provide emolliency, moisturization, or both, to the scalp. Suitable scalp conditioning agents include fatty alcohols, non-volatile hydrocarbons, low refractive index, nonvolatile polysiloxane fluids (below 1.46 refractive index) including those of the general structures represented above but characterized by lower or zero levels of aryl substitution, especially polydimethylsiloxane, and fatty esters and other lipid esters.

In one embodiment, the anti-dandruff composition or the hair care product of the invention is in the form of an emulsion (or dispersion), such as an oil-in-water or water-in-oil emulsion, particularly an oil-in-water emulsion.

The oil phase of the emulsion will preferably be mainly an emollient oil of the type used in personal care or cosmetic products. The emollient can and usually will be an oily material which is preferably liquid at ambient temperature. Alternatively, it can be solid at ambient temperature, in which case in bulk it will usually be a waxy solid, provided it is liquid at an elevated temperature at which it can be included in and emulsified in the composition.

Suitable normally liquid emollient oils include non-polar oils, medium polarity oils, synthetic oils, ether oils, and silicone oils.

The concentration of the oil phase may vary widely. The amount of the oil phase in the emulsion is preferably in the range from 0.5 wt. % to 80 wt. %, more preferably 1 wt. % to 30 wt. %, particularly 1.5 wt. % to 15 wt. %, and especially 2 wt. % to 10 wt. %, based on the total weight of the emulsion.

The amount of the aqueous phase in the emulsion is preferably in the range from 20 wt. % to 99.5 wt. %, more preferably 70 wt. % to 99 wt. %, particularly 85 wt. % to 98.5 wt. %, and especially 90 wt. % to 98 wt. %, based on the total weight of the emulsion.

A wide range of emulsifiers may be employed, particularly one or more cationic emulsifier(s). The specific nature of the emulsifier surfactant used in any particular instance depends on the type of emulsion being made, particularly the amount and nature of the oil being emulsified and the total desired level of emulsifier.

The concentration of emulsifier in the emulsion is preferably in the range from 0.1 wt. % to 20 wt. %, more preferably 0.5 wt. % to 15 wt. %, particularly 1 wt. % to 10 wt. %, and especially 2 wt. % to 7 wt. %, based on the total weight of the emulsion.

The emulsion suitably comprises in the range from 0.01 wt. % to 10 wt. %, preferably 0.5 wt. % to 5 wt. %, more preferably 0.1 wt. % to 4 wt. %, particularly 0.2 wt. % to 2 wt. %, and especially 0.3 wt. % to 1 wt. % of the anti-dandruff composition based on the total weight of the emulsion.

Many other components that may be used in hair care products or end-use products may also be combined with the anti-dandruff composition according to the present invention. These components may be oil soluble, water soluble or non-soluble. Examples of such materials include:
(i) preservatives such as those based on parabens (alkyl esters of 4-hydroxybenzoic acid), phenoxyethanol, substituted ureas and hydantoin derivatives, e.g. those sold commercially under the trade names Germaben II Nipaguard BPX and Nipaguard DMDMH, when used usually in a concentration in the range from 0.5 wt. % to 2 wt. % based on the total weight of the composition;
(ii) perfumes, when used typically at a concentration in the range from 0.1 wt. % to 10 wt. % more usually up to about 5 wt. % and particularly up to about 2 wt. %, based on the total weight of the composition;
(iii) humectants or solvents such as alcohols, polyols such as glycerol and polyethylene glycols, when used typically at a concentration in the range from 1 wt. % to 10 wt. % based on the total weight of the composition;
(iv) alpha hydroxy acids such as glycolic, citric, lactic, malic, tartaric acids and their esters; self-tanning agents such as dihydroxy acetone;
(v) vitamins and their precursors including: (a) Vitamin A, e.g. as retinyl palmitate and other tretinoin precursor molecules, (b) Vitamin B, e.g. as panthenol and its derivatives, (c) Vitamin C, e.g. as ascorbic acid and its derivatives, (d) Vitamin E, e.g. as tocopheryl acetate, (e) Vitamin F, e.g. as polyunsaturated fatty acid esters such as gamma-linolenic acid esters;
(vi) skin care agents such as ceramides either as natural materials or functional mimics of natural ceramides;
(vii) natural phospholipids, e.g. lecithin,
(viii) vesicle-containing formulations;
(ix) botanical extracts with beneficial skin care properties;
(x) skin whiteners such as kojic acid, arbutin and similar materials;
(xi) skin repair compounds actives such as Allantoin and similar series; (xii) caffeine and similar compounds;
(xiii) cooling additives such as menthol or camphor;
(xiv) insect repellents such as N,N-diethyl-3-methylbenzamide (DEET) and citrus or eucalyptus oils;
(xv) essential oils; and
(xvi) pigments, including microfine pigments, particularly oxides and silicates, e.g. iron oxide, particularly coated iron oxides, and/or titanium dioxide, and ceramic materials such as boron nitride, or other solid components, such as are used in make-up and cosmetics, to give suspoemulsions, typically used in an amount in the range from 1 wt. % to 15 wt. %, but usually at least 5 wt. %, and particularly about 10 wt. % based on the total weight of the composition.

Another aspect of the present invention relates to the use of an anti-dandruff composition as defined above or a hair care product as defined above for the treatment and/or prevention of dandruff.

In a particular embodiment, the use relates to a cosmetic use for the treatment and/or prevention of dandruff, i.e. to a non-therapeutical use.

In another aspect, the present invention relates to an anti-dandruff composition or a hair care product as defined above for use in the treatment and/or prevention of dandruff.

In another aspect, the present invention relates to a method of treatment and/or prevention of dandruff, the method comprising the application to a person in need thereof an anti-dandruff composition or a hair care product as defined above.

A further aspect of the invention provides a method of identifying a test compound or composition having use in treatment and/or prevention of dandruff, comprising
(a) assaying whether the test composition reduces Malassezia furfur growth, and/or,
(b) assaying whether the test composition reduces Malassezia globosa growth, and/or,
(c) assaying whether the test composition disrupts Malassezia restricta growth, wherein a compound or composition which is positive in one or more assays is of use in/as anti-dandruff compound or anti-dandruff composition.

In a particular embodiment, the method also comprises assaying whether the test composition reduces Staphylococcus aureus growth.

In a particular embodiment, the above-mentioned assays are disc-diffusion assays.

The method of the invention determines whether a test compound or composition is useful against dandruff. This is indicated by a positive result in one or more of the assays mentioned above. By “positive result”, it is meant that the compound or composition leads to an increased effect in the assay compared to a control reaction which includes the same sample mixture but lacking the test compound/composition.

In a particular embodiment, the anti-dandruff composition or the hair care product according to the present invention, respectively, further comprises one or more of an Epilobium extract, saccharide isomerate, Vitamin B6 and derivatives thereof, niacinamide, and panthenol (pro-vitamin B5).

The term Epilobum extract as used herein encompasses Epilobum extracts from any Epilobium species such as preferably from Epilobium fleischeri, Epilobium Parviflorum and/or Epliobium Angustifolium obtained from extraction of the respective Flowers, Leaves and/or Stems.

Suitable extracts encompass in particular Epilobium fleischeri and Epilobium Angustifolium extracts. Most preferred in all embodiments of the present invention is the use of an Epilobium fleischeri extract with all the definitions and preferences as given herein.

A particularly suitable Epilobium fleischeri extract according to the present invention is obtained by extraction of the Flowers/Leafs and Stems of the plants and is commercially available as ALPAFLOR® ALP-SEBUM from DSM Nutritional products, comprising 5-10-wt.-% of the extract.

An Epilobium Angustifolium Extract suitable for the purpose according to the present invention is commercially available as Defenscalp™ from Lucas Meyer Cosmetics.

An Epilobium Parviflorum Extract suitable for the purpose according to the present invention is commercially available from Kingherbs.

Generally, the amount of the Epilobium extract, preferably the Epilobium fleischeri extract (based on the extract) in the hair care products according to the present invention is selected in the range from 0.001 to 5 wt %, more preferably from 0.01 to 4 wt %, even more preferably from 0.1 to 3 wt %, furthermore preferably from 0.1 to 1 wt % and still more preferably from 0.1 to 0.5 wt %, based on the total weight of the composition or product. Further suitable ranges are from 0.05 to 1 wt.-% and from 0.05 to 0.5 wt.-%.

In case the diluted extract in the form of ALPAFLOR® ALP-SEBUM from DSM Nutritional Products Ltd is used, the amount is preferably selected in the range of 0.1-3 wt.-%, more preferably, 0.25 to 3 wt.-%, most preferably 0.5 to 3 wt.-%, based on the total weight of the composition or product.

In a particular embodiment, the anti-dandruff composition or the hair care product according to the present invention, respectively, further comprises saccharide isomerate. The term saccharide isomerate as used herein refers to a blend of sugars comprising psicose, mannose, fructose and glucose. In one embodiment, the blend of sugars consists essentially of psicose, mannose, fructose and glucose.

The saccharide isomerate according to the present invention may be prepared by isomerization of glucose, a process well known to a person skilled in the art. Preferably, the isomerization process comprises (a) dissolving glucose in water followed by (b) isomerization said glucose in the presence of a base, preferably in the presence of sodium hydroxide, more preferably at a temperature selected in the range from 25 to 100° C. and (c) purifying the resulting reaction mixture by chromatography and optionally filtration.

A particularly suited saccharide isomerate in all embodiments of the present invention is a saccharide isomerate consisting essentially of a) 1 to 5 wt.-percent, preferably 2 to 3 wt.-percent, of psicose, b) 1 to 5 wt.-percent, preferably 1.5 to 3 wt.-percent, of mannose, c) 10 to 30 wt.-percent, preferably 10 to 20 wt.-percent, of fructose, d) 15 to 30 wt.-percent, preferably 20 to 30 wt.-percent, of glucose, e) 0 to 5 wt.-percent, preferably 0 to 4 wt.-percent, of galactose, up 7.5 wt.-%, preferably up to 5 wt.-% of further sugars, 0.1 to 2 wt.-% of an additive, preferably citric acid and/or a salt thereof such as preferably the sodium salt, and up to 100% water.

The term consisting essentially of as used herein means that the total amount of the ingredients a) to g) ideally sums up to 100 wt.-%. It is however not excluded that small amount of unknown (sugar) impurities, e.g. derived from the isomerization process of glucose may be present.

A particularly suitable saccharide isomerate to be used in the anti-dandruff compositions respectively the hair care products according to the present invention is Pentavitin® which is commercially available from DSM Nutritional Products Ltd.

The total amount of saccharide isomerate to be incorporated into the anti-dandruff composition or hair care product according to the present invention, respectively, is preferably from 0.01 to 10 wt.-%, more preferably from 0.1 to 7.5 wt.-%, most preferably from 0.2 to 5 wt.-%, based on the total weight of the composition/product. Further suitable ranges are from 0.25 to 2.5 wt.-% and from 0.5 to 2 wt.-%. Particularly preferred ranges are from 0.2 to 1 wt.-%, more preferably from 0.25 to 0.75 wt.-%, such as from 0.3 to 0.6 wt.-%.

In a preferred embodiment, the saccharide isomerate such as in particular Pentavitin® is used together with at least one known anti-dandruff active as described above, such as even more in particular piroctone olamine.

A particularly preferred embodiment of the present invention relates to a hair care product that is a shampoo comprising the anti-dandruff composition according to the present invention with all the definitions and preferences as given herein and saccharide isomerate. Most preferred is a shampoo comprising Pentavitin® and piroctone olamine next to the anti-dandruff composition of the present invention. Even more preferably, the shampoo further comprises sodium laureth sufate and/or cocamidopropyl betaine, preferably both.

Niacinamide [CAS-Nr. 98-92-0] is one of the water-soluble B-complex vitamins which is e.g. available as Niacinamide PC or Niacinamide from DSM Nutritional Products AG, (4303 Kaiseraugst, Switzerland). Niacinamide is also referred to as nicotinamide or pyridine-3-carboxamide and is the amide of niacin (vitamin B3).

Preferably, the amount of niacinamide in the anti-dandruff compositions respectively hair care products according to the present invention is selected in the range of 0.5 to 10 wt. %, preferably in the range of 1 to 10 wt. %, most preferably in the range of 1 to 5 wt. %, based on the total weight of the composition/product.

The term Vitamin B6 and derivatives thereof refers in particular to pyridoxine hydrochloride [58-56-0], pyridoxal [CAS-Nr. 66-72-8] and pyridoxamin [CAS-Nr. 85-87-0]. In all embodiments particularly preferred is the use of pyridoxine hydrochloride also known as vitamin B6 hydrochloride or vitamin B6 which is e.g. available as Pyridoxine hydrochloride or Pyridoxine Hydrochloride 98 DC at DSM Nutritional Products AG, (4303 Kaiseraugst, Switzerland).

Preferably, the amount of Vitamin B6 or a derivative thereof such as in particular pyridoxine hydrochloride in the anti-dandruff compositions respectively hair care products according to the present invention is selected in the range of 0.02 to 6 wt. %, preferably in the range of 0.05 to 4 wt. %, most preferably in the range of 0.1 to 3 wt. %, based on the total weight of the composition/product.

Panthenol (INCI) is also referred to as D-panthenol, dexpanthenol, provitamin B5, or (+)-(R)-2,4-Dihydroxy-N-(3-hydroxypropyl)-3,3-dimethylbutyramid. Panthenol improves hydration, reduces itching and inflammation of the skin, improves skin elasticity, and accelerates epidermal wound healing. Panthenol is e.g. commercially as D-Panthenol at DSM Nutritional Products Europe Ltd.

In all embodiments of the present invention, the amount of panthenol used in the anti-dandruff compositions respectively hair care products compositions according to the present invention is advantageously selected in the range of 0.001 to 10 wt.-percent, preferably in the range of 0.01 to 7 wt-percent, more preferably in the range of 0.1 to 6 wt-percent and most preferably in the range of 0.5 to 5 wt-percent based on the total weight of the composition respectively product. Further preferred ranges are 0.5 to 4.5 wt-percent, 0.6 to 4 wt-percent, 0.6 to 3.5 wt-percent panthenol based on the total weight of the composition/product.

The invention will now be described in further detail by way of the following examples, which illustrate the benefits and advantages of the present invention.

### EXAMPLES

### Example 1: Determination of Antimicrobial Efficacy of Perfumery Materials by Minimal Inhibitory Concentration (MIC) Assay

The present inventors devised minimal inhibitory concentration (MIC) assay that can be used to determine the effectiveness of perfume ingredients or compositions thereof as anti-dandruff compositions.

The MIC assay for Malassezia furfur DSM 6170 was modified based on the method in Journal of Clinical Microbiology 2017, Vol 55: 1883-1893. In brief, working culture was prepared from stock cultures stored at −80° C. to obtain a cell suspension as the inoculum. Serial dilutions of samples, 29 to 10000 ppm, were tested to determine the MIC values for growth of Malassezia furfur DSM 6170 after at 30° C. for 48 h aerobically. Resazurin was used as growth indicator.

MIC assay for Staphylococcus aureus ATCC 6538 is based on Clinical Laboratory Standard Institute: CLSI/NCCLS M07-A8. Cell suspension of working cultures of S. aureus was prepared following the instructions of culture provider ATCC, USA. Serial dilutions of samples, 29 to 10000 ppm, were tested to determine the MIC values for growth of Staphylococcus aureus ATCC 6538 in tryptic soya broth (TSB) at 37° C. aerobically for 24 h.

A total of 26 perfumery materials were examined for an inhibition effect on Malassezia furfur and Staphylococcus aureus. The MIC values of those perfumery materials on M. furfur and S. aureus are shown in Table 1.

TABLE 1
MIC values of 26 perfumery materials on
S. aureus or/and Malassezia furfur

| | MIC (ppm) | MIC (ppm) |
|---|---|---|
| Chemical name | S. aureus | M. furfur |
| p-menthan-7-ol | 625 | 222 |
| (4E)-4-methyl-5-(4-methylphenyl)-4- | 75 | 333 |
| pentenal | | |
| 4-methyl-2-pentylpyridine | 693 | 222 |
| gamma-dodecalactone | 173 | 148 |
| methyl 2,4-dihydroxy-3,6-dimethylbenzoate | 111 | 290 |
| (2E)-2-dodecenal | 49 | 148 |
| 2-ethyl-1-hexanol | 867 | 44 |
| citral | 450 | 222 |
| cyclopropylmethyl (3Z)-3-hexenoate + | 625 | 333 |
| cyclopropylmethyl (3E)-3-hexenoate | | |
| floral butanal | 387 | 1300 |
| (2E,6Z)-2,6-nonadienal | 233 | 44 |
| cinnamaldehyde | 293 | 99 |
| para-anisaldehyde | | 500 |
| cyclamen aldehyde | 933 | |
| indole | 693 | 259 |
| ethyl vanillin | 2667 | 500 |
| 1-decanol | 370 | 500 |
| geranium rose oil | 697 | 500 |
| litsea cubeba oil | 900 | 148 |
| delta-undecalactone | 293 | 333 |
| methyl dihydrojasmonate | 330 | 222 |
| Lemongrass | | 333 |
| cinnamon oil | 257 | |
| para-anisyl propanal | 530 | 222 |
| lilial | 325 | |
| 1-dodecanol | 22 | |

### Example 2: Determine Anti-Malassezia Effect of Perfumery Materials in Solvent Using Disk-Diffusion Assay

Anti-Malassezia effect of perfumery materials in solvent in DMSO was determined using disc-diffusion assay, based on the method of J Mycol Infect 2018: 23: 91-98. In brief, working cultures of Malassezia strains were prepared from −80 C stock culture on mDixon agar plate (for M. furfur DSM 6170) or mLN agar plate (for M. restricta ATCC MYA-4611 and M. globosa ATCC MYA-4612), and cells were suspended in 0.9% saline, and spread onto mDixon or mLN agar surface to prepare lawn plate. Test samples solutions are prepared in DMSO to final concentrations of 0.2%, 1% or 2%. Discs (Whatman, 6 mm in diameter) were placed on inoculated agar plates with each Malassezia strains respectively, including M. furfur DSM 6170, M. restricta ATCC MYA-4611, and M. globosa ATCC MYA-4612. Aliquots (20 μL) of each sample solution was added to each disc. Agar plates were incubated at 30° C. for 3 days for M. furfur DSM 6170, and 6 days for M. restricta ATCC MYA-4611, and M. globosa ATCC MYA-4612. After incubation, diameters of the clear inhibition zones around the discs were measured. Inhibition zone greater than 6 mm are regarded as having anti-Malassezia effect.

The inhibition zones of 31 perfumery materials at concentrations of 0.2%, 1% or 2% were shown in Table 2. The anti-Malassezia effect of those materials are dose dependent and strain dependent.

TABLE 2
Disc diffusion assay results of 31 perfumery materials
in DMSO on Malassezia strains

| | Inhibition zone (mm) | | | | | | |
|---|---|---|---|---|---|---|---|
| | (NI means no inhibition zone) | | | | | | |
| | M. furfur | M. restricta | | | M. globosa | | |
| Chemical name | 2% | 0.2% | 1% | 2% | 0.2% | 1% | 2% |
| p-menthan-7-ol | 6.3 | 6.7 | | | 6.5 | | |
| ethyl 2-methyl | | | NI | | | 6.6 | |
| butanoate | | | | | | | |
| 1-hexanol | | | 6.7 | | | NI | |
| (4E)-4-methyl-5- | 7.5 | 6.8 | 11.6 | 12.0 | | 9.0 | 10.5 |
| (4-methylphenyl)- | | | | | | | |
| 4-pentenal | | | | | | | |
| 4-methyl-2- | 6.7 | 6.6 | | | 6.7 | | |
| pentylpyridine | | | | | | | |
| methyl octine | | 6.4 | 6.7 | | NI | 10.3 | |
| carbonate | | | | | | | |
| gamma- | 6.9 | NI | | | 6.3 | | |
| dodecalactone | | | | | | | |
| methyl 2,4- | 7.0 | 6.3 | | 12.0 | 7.4 | | 9.2 |
| dihydroxy-3,6- | | | | | | | |
| dimethylbenzoate | | | | | | | |
| lilial | NI | | NI | | | 7.4 | |
| (2E)-2-dodecenal | 12.6 | 10.0 | 14.4 | 18.1 | | 15.2 | 17.9 |
| cinnamaldehyde | 16.5 | 8.4 | 34.5 | 33.3 | | 17.6 | 27.4 |
| 2-ethyl-1-hexanol | 7.6 | | 6.4 | | | 6.7 | |
| Cyclopropylmethyl | 8.0 | 6.3 | | 11.0 | 8.7 | | 13.0 |
| (3Z)-3-hexenoate + | | | | | | | |
| cyclopropylmethyl | | | | | | | |
| (3E)-3-hexenoate | | | | | | | |
| floral butanal | NI | | 8.2 | | | NI | |
| cyclamen | NI | | 8.2 | | | 6.7 | |
| aldehyde | | | | | | | |
| citral | 8.6 | 6.2 | 7.1 | >40 | 6.2 | | 8.7 |
| (2E,6Z)-2,6- | 13.5 | 7.7 | >40 | >40 | 12.4 | >40 | |
| nonadienal | | | | | | | |
| cinnamon oil | 11.4 | 7.7 | 20.6 | 38.2 | | 20.0 | 19.0 |
| 1-dodecanol | | 6.8 | 11.5 | | 8.7 | 10.2 | |
| cyclamen | NI | 6.6 | | 12.3 | 6.3 | | 7.9 |
| propanal | | | | | | | |
| 10-undecenal | | 6.5 | 12.7 | | 7.1 | 14.1 | |
| para- | NI | | NI | 13.4 | | NI | NI |
| anisaldehyde | | | | | | | |
| para-anisyl | NI | 6.6 | 14.3 | | NI | 11.6 | |
| propanal | | | | | | | |
| indole | NI | NI | NI | | 6.3 | 15.2 | |
| ethyl vanillin | NI | | NI | 19.5 | | NI | NI |
| 1-decanol | NI | 8.5 | 10.0 | 25.0 | 6.7 | 15.9 | 16.4 |
| geranium rose oil | NI | 6.5 | | | 6.3 | | |
| litsea cubeba oil, | NI | 6.3 | | | 6.4 | | |
| delta- | NI | 6.1 | | | 6.4 | | |
| undecalactone | | | | | | | |
| methyl | NI | 6.1 | | | NI | NI | |
| dihydrojasmonate | | | | | | | |
| lemongrass | | NI | | | 6.3 | | |

### Example 3: Determine Anti-Malassezia Effect of Mixtures of Perfumery Materials in Solvent Using Disc-Diffusion Assay

Table 3 shows the formulations of four perfumery material mixtures.

TABLE 3
Mixtures of perfumery materials

| | Mixtures (w/w %) | | | |
|---|---|---|---|---|
| Chemical Name | Mix 1 | Mix 2 | Mix 3 | Mix 4 |
| 1-dodecanol | 55.5 | 75.2 | | 53.7 |
| 1-decanol | 40.0 | | 17.8 | 20.0 |
| p-menthan-7-ol | | 20.0 | 30.0 | |
| gamma-dodecalactone | | | 10.0 | |
| methyl dihydrojasmonate | | | 30.0 | |
| Geranium rose oil | | | 5.0 | |
| cinnamaldehyde | | | | 5.0 |
| citral | | | | 20.0 |

| | |
|---|---|
| Perfuming ingredients | add up to 100% |

Anti-Malassezia effect of perfumery material mixtures in solvent DMSO was determined by disc-diffusion assay, based on the method of J Mycol Infect 2018; 23: 91-98. In brief, working cultures of M. restricta ATCC MYA-4611 were prepared from −80 C stock culture on mLN agar plate, and cells were suspended in 0.9% saline, and spread onto mLN agar surface to prepare lawn plate. Test sample solutions are prepared in DMSO to final concentration of 0.5%. Discs (Whatman, 6 mm in diameter) were placed on inoculated agar plates with M. restricta. Aliquots (20 μL) of each sample solution was added to each disc. Agar plates were incubated at 30° C. for 6 days. After incubation, diameters of the clear inhibition zones around the discs were measured. Inhibition zone greater than 6 mm are regarded as having anti-Malassezia effect.

Table 4 shows that the mixtures (mix 2, mix 3, and mix 4) show inhibition effect on M. restricta.

TABLE 4
Disc diffusion assay results of four perfumery
material mixtures on M. restricta

| Mixtures | Inhibition zone (mm) M. restricta |
|---|---|
| Mix 2 | 6.4 |
| Mix 3 | 6.4 |
| Mix 4 | 6.1 |

### Example 4: Anti-Malassezia Effect of Perfumery Materials in Combination of Anti-Dandruff Actives in Solvent

The anti-Malassezia effect of perfumery materials in combination of anti-dandruff actives using the disk-diffusion assay, modified based on the method of J Mycol Infect 2018; 23: 91-98. working culture of M. furfur DSM 6170 was prepared from −80 C stock culture on mDixon agar plate, and cells were suspended in 0.9% saline, and spread onto mDixon agar surface to prepare lawn plates. Discs (Whatman, 6 mm in diameter) were placed on inoculated mDixon agar plates. Test sample solutions are prepared in DMSO. Aliquots (20 μL) of each sample solution was added to each disc. Agar plates were incubated at 30° C. for 3 days. After incubation, diameters of the clear inhibition zones around the discs were measured.

Anti-Malassezia effect of combination of (2E,6Z)-2,6-nonadienal with anti-dandruff actives

Disc diffusion results of (2E,6Z)-2,6-nonadienal alone and in combination with various anti-dandruff actives in DMSO against M. furfur are tested.

Table 5 shows that binary mixture of 0.19% Piroctone Olamine and 0.75% (2E,6Z)-2,6-nonadienal had higher anti-Malassezia efficacy than 0.19% PO and 0.75% (2E,6Z)-2,6-NONADIENAL individual anti-Malassezia efficacy.

Table 6 shows that binary mixture of 0.5% salicylic acid and 1% (2E,6Z)-2,6-nonadienal showed higher anti-Malassezia efficacy than 0.5% salicylic acid and 1% (2E,6Z)-2,6-nonadienal individual anti-malassezia efficacy.

Table shows that binary mixture of 0.1%-0.4% ketoconazole and 1% (2E,6Z)-2,6-nonadienal had higher anti-Malassezia efficacy than 0.1%-0.4% ketoconazole and 1% (2E,6Z)-2,6-nonadienal individual anti-Malassezia efficacy.

TABLE 5
Disc diffusion assay results of (2E,6Z)-2,6-nonadienal,
Piroctone Olamine, and binary mixtures

| | Inhibition |
|---|---|
| | zone (mm) |
| Composition | on M. furfur |
| 0.19% Piroctone Olamine (PO) | 11.8 |
| 0.75% (2E,6Z)-2,6-nonadienal | 7.6 |
| 0.75% (2E,6Z)-2,6-nonadienal + | 12.8 |
| 0.19% PO | |

TABLE 6
Disc diffusion results of (2E,6Z)-2,6-nonadienal, salicylic
acid and binary mixtures against M. furfur

| | Inhibition |
|---|---|
| | zone (mm) |
| Composition | on M. furfur |
| 0.5% salicylic acid | NI |
| 1% (2E,6Z)-2,6-nonadienal | 11.4 |
| 0.5% salicylic acid + | 13.7 |
| 1% (2E,6Z)-2,6-nonadienal | |

TABLE 7
Disc diffusion results of (2E,6Z)-2,6-nonadienal, ketoconazole
and binary mixtures against M. furfur

| | Inhibition |
|---|---|
| | zone (mm) |
| Compositions | on M. furfur |
| 0.1% ketoconazole | 22.3 |
| 0.2% ketoconazole | 24.6 |
| 0.3% ketoconazole | 24.5 |
| 0.4% ketoconazole | 24.9 |
| 1% (2E,6Z)-2,6-nonadienal | 14.1 |
| 0.1% ketoconazole + | 24.5 |
| 1% (2E,6Z)-2,6-nonadienal | |
| 0.2% ketoconazole + | 29.4 |
| 1% (2E,6Z)-2,6-nonadienal | |
| 0.3% ketoconazole + | 30.5 |
| 1% (2E,6Z)-2,6-nonadienal | |
| 0.4% ketoconazole + | 28.1 |
| 1% (2E,6Z)-2,6-nonadienal | |

### Example 5: Anti-Malassezia Effect of Perfumery Materials and Mixtures in Shampoo Bases

The present inventors sought to investigate how whether the anti-dandruff effect of compounds and compositions could be used in shampoo formulations. For this reason, they prepared two shampoo bases. Shampoo Base A is a composition with Sodium Laureth Sulfate and cocamidopropyl betaine as the primary ingredients, while Shampoo Base B is a composition with Ammonium Lauryl Sulfate and Ammonium Laureth Sulfate as primary ingredients.

Anti-Malassezia effect of perfumery materials in shampoo was determined using disc-diffusion assay, based on the method of J Mycol Infect 2018; 23: 91-98. In brief, working cultures of M. restricta ATCC MYA-4611 or M. globosa ATCC MYA-4612 were prepared from −80 C stock culture on mLN agar plate, and cells were suspended in 0.9% saline, and spread onto mLN agar surface to prepare lawn plate. Test sample solutions of perfumery materials are prepared using 25% Shampoo Base solutions in Milli Q to final concentrations of 0.25%. Discs (Whatman, 6 mm in diameter) were placed on inoculated agar plates with each Malassezia strains respectively, including M. restricta ATCC MYA-4611, and M. globosa ATCC MYA-4612. Aliquots (20 μL) of each sample solution was added to each disc. Agar plates were incubated at 30° C. for 6 days. After incubation, diameters of the clear inhibition zones around the discs were measured. Inhibition zone greater than 6 mm are regarded as having anti-Malassezia effect.

Table 8 shows that 0.25% (2E,6Z)-2,6-NONADIENAL, 2-methoxy-4-(2-propen-1-yl)phenol and 3-Propylphenol in Base B show inhibition effect on both M. restricta and M. globosa.

TABLE 8
Anti-Malassezia effect of perfumery materials
in Shampoo Base B using Disc-diffusion assay

| 0.25% perfumery | | |
|---|---|---|
| materials in | Inhibition | Inhibition |
| 25% Shampoo | zone (mm) | zone (mm) |
| Base B solution | M. restricta | M. globosa |
| (2E,6Z)-2,6-nonadienal | 11.1 | 12.8 |
| 2-methoxy-4-(2-propen- | 7.3 | 8.2 |
| 1-yl)phenol | | |
| 3-propylphenol | 7.7 | 10.2 |

Anti-Malassezia effect of perfumery materials in shampoo was determined using Cylinder diffusion assay. Working cultures of M. restricta ATCC MYA-4611 and inoculated as described in disc diffusion assay. Cylinders were placed on inoculated agar plates. Aliquots (80 μL) of each sample solution were added to each cylinder. Agar plates were incubated at 30° C. for 6 days. After incubation, diameters of the clear inhibition zones around the discs were measured. Inhibition zone greater than 6 mm are regarded as having anti-Malassezia effect.

Table 9 shows that mixtures of mix 1, mix 2, mix 3 and mix 4 at concentration of 0.25% in shampoo base A had inhibition effect on M. restricta.

TABLE 9
Anti-Malassezia effect of perfumery material mixtures
in shampoo bases using Cylinder-diffusion assay

| 0.25% perfumery | |
|---|---|
| material mixtures | Inhibition |
| in 25% Shampoo | zone (mm) |
| Base A solution | M. restricta |
| Without mixtures | 13.1 |
| Mix 1 | 26.5 |
| Mix 2 | 26.6 |
| Mix 3 | 27.4 |
| Mix 4 | 30.7 |

### Example 6 Additional Mixtures of Perfumery Materials

Table 10 shows the formulation of additional perfumery material mixtures with anti-Malassezia efficacy

TABLE 10
Mixtures of perfumery materials

| | Mixtures (w/w %) | | | | |
|---|---|---|---|---|---|
| Chemical name | Mix 5 | Mix 6 | Mix 7 | Mix 8 | Mix 9 |
| cinnamaldehyde | 30 | 44 | | | |
| (2E,6Z)-2,6-NONADIENAL | | | | 18.5 | |
| LEMONGRASS | | | 10 | | |
| CINNAMON OIL | | | 50 | 10 | |
| citral | 40 | 11 | | | 60 |
| delta-undecalactone | | | | 21.5 | 30 |
| LITSEA CUBEBA | | | | 50 | |
| BERRY OIL | | | | | |
| 10-undecenal | 2 | 4 | | | |
| p-menthan-7-ol | 23 | 38 | | | 10 |
| cyclopropylmethyl | 5 | 3 | 40 | | |
| (3Z)-3-hexenoate + | | | | | |
| cyclopropylmethyl | | | | | |
| (3E)-3-hexenoate | | | | | |

### Example 7 Anti-Malassezia Effect of Additional Mixtures of Perfumery Materials

Anti-Malassezia effect of perfumery materials in shampoo was determined using Cylinder diffusion assay. Working cultures of M. restricta ATCC MYA-4611 and inoculated as described in disc diffusion assay. Cylinders were placed on inoculated agar plates. Aliquots (80 μL) of each sample solution were added to each cylinder. Agar plates were incubated at 30° C. for 6 days for. After incubation, diameters of the clear inhibition zones around the discs were measured. Inhibition zone greater than 6 mm are regarded as having anti-Malassezia effect.

Table 11 shows that mixtures of mix 5, mix 6, mix 7, mix 8, and mix 9 at concentration of 0.25% in shampoo base A had inhibition effect on M. restricta

TABLE 11
Anti-Malassezia effect of perfumery material mixtures
in shampoo bases using Cylinder-diffusion assay

| 0.25% perfumery | |
|---|---|
| material mixtures | Inhibition |
| in 25% Shampoo | zone (mm) |
| Base A solution | M. restricta |
| Without mix | 14.8 |
| Mix 5 | 33.0 |
| Mix 6 | >40 |
| Mix 7 | >40 |
| Mix 8 | >40 |
| Mix 9 | >40 |

### Example 8: Effect of Perfumery Materials on the Anti-Malassezia Activities of Piroctone Olamine (PO) in DMSO

Anti-Malassezia effect of combinations of PO and perfumery materials in solvent DMSO against M. restricta was determined using disc-diffusion assay as described above. Aliquots (20 μL) of each sample solution was added to each disc. Agar plates were incubated at 30° C. for 4 days. After incubation, diameters of the clear inhibition zones around the discs were measured. Ratio of inhibition zone diameter of each combination to that of PO only were calculated. When the ratio is greater than 1.0, it is regarded that perfumery materials have boosting effect on the anti-Malassezia activity of PO.

Table 12 shows that the perfumery raw materials have boosting effect on the anti-Malassezia activity of 0.1% or 0.35% PO in DMSO.

TABLE 12
Anti-Malassezia test results of combination of perfumery materials
with piroctone olamine (PO) in DMSO against M. restricta

| | Ratio of inhibition zone diameter of combination | | | |
|---|---|---|---|---|
| | of perfumery materials and PO to that of PO only | | | |
| | at the corresponding concentrations of PO | | | |
| | (0.2% PRM + | (0.2% PRM + | (0.5% PRM + | (1% PRM + |
| Perfumery raw | 0.1% PO) | 0.35% PO) | 0.1% PO) | 0.35% PO) |
| materials (PRM) | to 0.1% PO | to 0.35% PO | to 0.1% PO | to 0.35% PO |
| (E)-3-phenyl-2-propenal | 1.4 | 1.3 | 3.4 | |
| (2E,6Z)-2,6-nonadienal | 1.2 | | 3.4 | |
| cinnamon oil ceylon | 1.5 | 1.2 | 3.2 | |
| (E)-3-phenyl-2-propenal + | 1.4 | 1.1 | 3.1 | |
| 2-methoxy-4-(2-propen-1- | | | | |
| yl)phenol | | | | |
| (2E)-2-dodecenal | | | 2.1 | 1.2 |
| (+/−)-3- | | | 2.0 | 1.5 |
| (4-methoxyphenyl)-2- | | | | |
| methylpropanal | | | | |
| (4E)-4-methyl-5-(4- | | | 1.2 | |
| methylphenyl)-4-pentenal | | | | |
| nonanoic acid | | | 1.1 | 1.2 |
| octanoic acid | | | | 1.1 |
| decanoic acid | | | | 1.1 |
| (2Z)-3,7-dimethyl- | | | | 1.1 |
| 2,6-octadienal + | | | | |
| (2E)-3,7-dimethyl- | | | | |
| 2,6-octadienal | | | | |

### Example 9: Mixtures with Anti-Malassezia Perfumery Materials Showing Boosting Effect on the Anti-Malassezia Activities of Piroctone Olamine (PO) in DMSO

The following perfumery material mixtures were created with 11 anti-Malassezia perfumery materials (see Table 13).

| | Mix | Mix | Mix | Mix | Mix | Mix | Mix | Mix | Mix | Mix | Mix | Mix | Mix |
|---|---|---|---|---|---|---|---|---|---|---|---|---|---|
| Chemical Name | 10 | 11 | 12 | 13 | 14 | 15 | 16 | 18 | 19 | 20 | 21 | 22 | 23 |
| (2E,6Z)-2,6- | 0.05% | 0.10% | 0.05% | 0.10% | | | | 0.10% | | 0.05% | | | |
| nonadienal | | | | | | | | | | | | | |
| (E)-3-phenyl-2- | 0.05% | 0.05% | 0.10% | 0.05% | | 0.10% | | | | 0.15% | 0.10% | 0.10% | 0.10% |
| propenal | | | | | | | | | | | | | |
| cinnamon oil | 0.05% | | | | | | 0.15% | | | | | | |
| ceylon | | | | | | | | | | | | | |
| (E)-3-phenyl-2- | 0.05% | | | | | | | | | | | | |
| propenal + 2- | | | | | | | | | | | | | |
| methoxy-4-(2- | | | | | | | | | | | | | |
| propen-1-yl)phenol | | | | | | | | | | | | | |
| (2E)-2-dodecenal | | | 0.40% | | 0.25% | 0.20% | 0.25% | 0.25% | 0.15% | | 0.20% | 0.20% | |
| (+/−)-3-(4- | | 0.45% | | | 0.10% | 0.20% | 0.20% | | | | 0.20% | | 0.20% |
| methoxyphenyl)-2- | | | | | | | | | | | | | |
| methylpropanal | | | | | | | | | | | | | |
| (4E)-4-methyl-5- | | | | 0.45% | 0.10% | | | | 0.15% | | | 0.20% | 0.20% |
| (4-methylphenyl)- | | | | | | | | | | | | | |
| 4-pentenal | | | | | | | | | | | | | |
| nonanoic acid | | | | | | | | 0.25% | 0.15% | | | | |
| octanoic acid | | | | | 0.55% | 0.50% | 0.40% | | | | | | |
| decanoic acid | | | | | | | | 0.40% | | | | | |
| (2Z)-3,7-dimethyl- | | 0.40% | 0.45% | 0.40% | | | | | 0.55% | | | | |
| 2,6-octadienal + | | | | | | | | | | | | | |
| (2E)-3,7-dimethyl- | | | | | | | | | | | | | |
| 2,6-octadienal | | | | | | | | | | | | | |
| Total dosage of | 0.2% | 1.0% | 1.0% | 1.0% | 1.0% | 1.0% | 1.0% | 1.0% | 1.0% | 0.2% | 0.5% | 0.5% | 0.5% |
| PRM mixtures | | | | | | | | | | | | | |

Anti-Malassezia effect of combinations of PO and perfumery raw materials mixtures in solvent DMSO was determined using disc-diffusion assay, as described above. Aliquots (20 μL) of each sample solution was added to each disc. Agar plates were incubated at 30° C. for 4 days. After incubation, diameters of the clear inhibition zones around the discs were measured. Ratio of inhibition zone diameter of each combination to that of PO only were calculated. When the ratio is greater than 1.0, it is regarded that perfumery materials have boosting effect on the anti-Malassezia activity of PO.

Table 14 shows that combinations of PO with the PRM mixtures, at dosages of 0.2%, 0.5% and 1%, had greater anti-Malassezia effect than PO, as indicated by ration >1. This means that the mixtures have boosting effect on the anti-Malassezia activity of PO.

TABLE 14
Anti-Malassezia test results of combinations of piroctone
olamine (PO) and perfumery raw material (PRM) mixtures
in DMSO on M. restricta using disc assay.

| | | Ratio of inhibition zone diameter | |
|---|---|---|---|
| | | (mm) of combinations of | |
| | | PO and PRM mixtures to that of PO | |
| | Dosage of | (PRM mixtures + | (PRM mixtures + |
| Code of PRM | PRM | 0.1% PO) to | 0.35% PO) to |
| mixtures | mixtures | 0.1% PO | 0.35% PO |
| Mix 10 | 0.2% | n.a. | 1.1 |
| Mix 11 | 1.0% | 1.9 | 1.4 |
| Mix 12 | 1.0% | 2.1 | 1.4 |
| Mix 13 | 1.0% | 1.5 | 1.1 |
| Mix 14 | 1.0% | 1.3 | n.a. |
| Mix 15 | 1.0% | 1.6 | 1.1 |
| Mix 16 | 1.0% | 1.9 | 1.2 |
| Mix 18 | 1.0% | 1.3 | n.a. |
| Mix 19 | 1.0% | 1.4 | 1.1 |
| Mix 20 | 0.2% | 1.2 | 1.1 |
| Mix 21 | 0.5% | 1.6 | 1.1 |
| Mix 22 | 0.5% | 1.4 | 1.1 |
| Mix 23 | 0.5% | 1.5 | 1.1 |

### Example 10: Effect of Perfumery Material Mixtures on Anti-Malassezia Activity of Shampoo Base a Containing PO

Base A is a composition with sodium laureth sulfate and cocamidopropyl betaine as the primary ingredients.

Anti-Malassezia effect of shampoos was determined using cylinder diffusion assay as described above. Ratio of inhibition zone diameter of shampoo base A containing PO and perfumery material mixtures to that of shampoo base A containing PO only were calculated. Perfumery material mixtures were regarded as having boosting effect when the ratio is greater than 1.0.

Table 15 shows the boosting effect (ratio >1) of perfumery materials mixtures at 1% or 2% on the anti-Malassezia activity of shampoo base A with 0.35% PO against M. restricta.

TABLE 15
Anti-Malassezia test results of 25% shampoos containing PO
and perfumery material mixtures against M. restricta

| | |
|---|---|
| | Ratio of shampoos containing |
| | 0.35% PO and perfumery |
| Code of | material mixtures against shampoo |
| perfumery | base A with 0.35% PO only |

| material | 1% perfumery | 2% perfumery |
|---|---|---|
| mixtures | material mixture | material mixture |
| Mix 11 | 1.1 | 1.3 |
| Mix 12 | n.a. | 1.2 |
| Mix 15 | 1.1 | 1.2 |
| Mix 16 | 1.1 | 1.3 |

### Example 11: Anti-Malassezia Effect of Perfumery Material Mixtures in Hair Conditioner Base

A hair conditioner base was prepared. The main components included water, cetyl alcohol, MIRASIL® ADME-NP (water (and) amodimethicone (and) trideceth-6), stearyl alcohol, behentrimonium methosulfate (and) Isopropanol.

Anti-Malassezia effect of hair conditioners was determined using cylinder diffusion assay that was used for testing shampoos, as described above. In brief, working cultures of M. restricta ATCC MYA-4611 and inoculated as described in disc diffusion assay. Cylinders were placed on inoculated agar plates. Aliquots (80 μL) of each sample (25% conditioner solution) were added to each cylinder. Agar plates were incubated at 30° C. for 4 days. After incubation, diameters of the clear inhibition zones around the discs were measured. No inhibition zone is regarded as having no anti-Malassezia effect.

Table 16 shows four hair conditioners with 0.35% PO and perfumery materials mixtures have anti-Malassezia effect, in contrast, hair conditioner with 0.35% PO only has no anti-Malassezia effect.

TABLE 16
Anti-Malassezia test results of 25% conditioner
base containing 0.35% PO with/without perfumery
material mixtures against M. restricta

| Conditioners containing | Inhibition zone |
|---|---|
| 0.35% PO with/without | diameter (mm) |
| perfumery material mixtures | against M. restricta |
| Without Mixture | No inhibition zone |
| 2% Mix 11 | 9.9 |
| 2% Mix 12 | 13.8 |
| 2% Mix 15 | 12.9 |
| 2% Mix 16 | 14.4 |

### Example 12: Anti-Malassezia Effect of Mixtures in Shampoo Base on M. furfur

Anti-Malassezia effect of mixtures of perfumery materials in base A was determined using cylinder-diffusion assay, based on the method of J Mycol Infect 2018; 23: 91-98. In brief, working culture of M. furfur DSM 6170 was prepared from −80° C. stock culture on mDixon agar plate, and cells were suspended in 0.9% saline, and spread onto mDixon agar surface to prepare lawn plate. Cylinders were placed on inoculated agar plates. Aliquots (80 μL) of each sample solution (25% shampoo solution) were added to each cylinder. Agar plates were incubated at 30° C. for 3 days. After incubation, diameters of the clear inhibition zones around the discs were measured. Ratio of inhibition zone diameter of shampoo base A with perfumery material mixtures to that of shampoo base A were calculated. Perfumery material mixtures were regarded as having additional anti-Malassezia effect when the ratio is greater than 1.0.

Table 17 shows that seven mixtures, mix 5, mix 6, mix 7, mix 8, mix 11, mix 12, and mix 13, at concentration of 1% or/and 2%, in shampoo base A had additional anti-Malassezia effect on top of that of shampoo base only (ratio >1).

TABLE 17
Anti-Malassezia test results of 25% shampoo base A with
perfumery material mixtures against M. furfur DSM 6170

| | |
|---|---|
| | Ratio of inhibition zone diameter |
| | of shampoo base A with perfumery |
| Perfumery | material mixtures to that |
| material | of shampoo base A |

| mixture | 1% Mixture | 2% Mixture |
|---|---|---|
| Mix 5 | 1.3 | 1.8 |
| Mix 6 | 1.7 | 2.5 |
| Mix 7 | 1.2 | 1.7 |
| Mix 8 | 1.1 | 1.3 |
| Mix 11 | 1.1 | 1.2 |
| Mix 12 | n.a. | 1.2 |
| Mix 13 | n.a. | 1.2 |

### Example 13: In Vivo Test of Perfumery Material Mixture in Shampoo Base

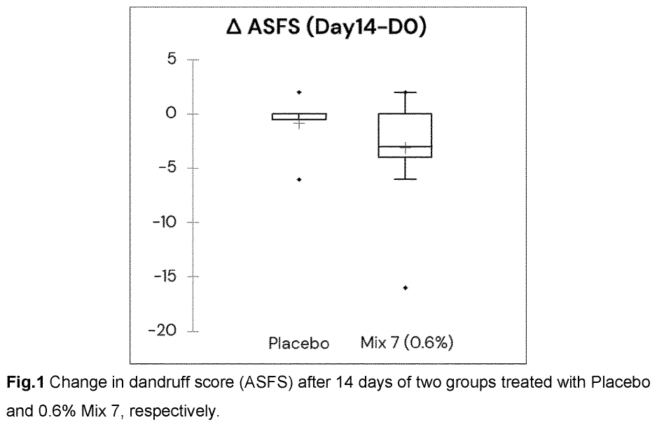

Anti-dandruff efficacy of a shampoo product containing one perfumery material mixture, 0.6% Mix 7, in shampoo base A was tested in an in vivo test. Shampoo base A itself was used as placebo. Product and placebo, two groups in total. 16 subjects with initial dandruff score, i.e. adherent scalp flaking score (ASFS)≥24, were recruited for each group. After a two weeks of wash-out period using no anti-dandruff claimed commercial shampoo, the subjects were required to wash their hair and scalp using the products three times a week. ASFS was evaluated for each subject at Day 0 and Day 14 during the in vivo test. The group treated with 0.6% Mixture 7 showed significant ASFS decrease at Day 14 comparing to Day 0, whereas the placebo group showed no significant ASFS changes (FIG. 1).

### Example 14: Anti-Malassezia Effect of Compounds in DMSO Using Disc-Diffusion Assay

Anti-Malassezia effect of additional perfumery materials in DMSO was determined using disc-diffusion assay as described previously. Test samples solutions were prepared in DMSO to final concentrations of 0.5%, 1% or 2%. Discs (Whatman, 6 mm in diameter) were placed on inoculated agar plates. Aliquots (20 μL) of each sample solution was added to each disc. Agar plates were incubated at 30° C. for 4 days. After incubation, diameters of the clear inhibition zones around the discs were recorded

Table 18 shows that the tested compounds had anti-Malassezia effect at concentrations of 0.5%, 1% or 2%.

TABLE 18
Anti-Malassezia test results of the tested
compounds in DMSO against M. restricta

Inhibition zone diameter (mm)

| Chemical name | 0.5% | 1% | 2% |
|---|---|---|---|
| (+/−)-(4E)-2,4-dimethyl-5- | 7.8 | | |
| (4-methylphenyl)-4-pentenal | | | |
| 3-[4-(2-hydroxy-2- | NI (no inhibition | 8.9 | 16.0 |
| methylpropyl)phenyl]propanal | zone) | | |

### Example 15: Anti-Malassezia Effect of Additional Perfumery Materials in DMSO

Anti-Malassezia effect of additional perfumery materials in DMSO was determined using disc-diffusion assay as described above. Test samples solutions were prepared in DMSO to final concentrations of 0.5%, 1% or 2%. Discs (Whatman, 6 mm in diameter) were placed on inoculated agar plates. Aliquots (20 μL) of each sample solution was added to each disc. Agar plates were incubated at 30° C. for 4 days. After incubation, diameters of the clear inhibition zones around the discs were recorded.

Table 19 shows that the following 14 perfumery materials have anti-Malassezia effect.

TABLE 19
Anti-Malassezia test results of 14 perfumery
materials in DMSO against M. restricta

| | Inhibition zone |
|---|---|
| Chemical name | diameter (mm) |
| (2E,4E)-2,4-heptadienal | >40 |
| (E,E)-2,4-decadienal | >40 |
| (E,E)-2,6-nonadienal | >40 |
| (E)-2-undecenal | >40 |
| (Z)-2-decenal | >40 |
| (2E)-2,4-undecadienal | 26.6 |
| (2E)-2,4-dodecadienal | 21.5 |
| (4Z)-4-dodecenal | 7.4 |
| (+/−)-(4Z)-4,8-dimethyl-4,9- | 7.3 |
| decadienal + (+/−)- | |
| (4E)-4,8-dimethyl-4,9-decadienal | |
| (Z)-4-decenal | 7.2 |
| (E)-2-nonen-1-ol | 7.5 |
| (−)-(3S)-3,7-dimethyl-6-octen- | 7.3 |
| 1-ol + (E)-3,7-dimethyl-2,6- | |
| octadien-1-ol | |
| (Z)-5-octen-1-ol | 7.3 |
| (Z)-3-octen-1-ol | 7.1 |

### Example 16: Anti-Malassezia Effect of Additional Perfumery Materials in DMSO

Anti-Malassezia effect of additional perfumery materials in solvent in DMSO was determined using disc-diffusion assay, as described previously. Test samples solutions are prepared in DMSO to final concentrations of 0.5%. Discs (Whatman, 6 mm in diameter) were placed on inoculated agar plates. Aliquots (20 μL) of each sample solution was added to each disc. Agar plates were incubated at 30° C. for 4 days. After incubation, diameters of the clear inhibition zones around the discs were measured.

TABLE 20
Anti-Malassezia test results of perfumery materials in DMSO against M. restricta

| | Inhibition zone |
|---|---|
| | diameter (mm) on |
| Chemical name | M. restricta |
| (E)-2-nonenal | 38.0 |
| (E)-2-octenal | 24.8 |
| (+/−)-3-(1,3-benzodioxol- | 19.0 |
| 5-yl)-2-methylpropanal | |
| (E)-2-tridecenal | 15.2 |
| (4-methylphenyl)acetaldehyde | 10.6 |
| 4-formyl-2-methoxyphenyl | 10.2 |
| 2-methylpropanoate | |
| (+/−)-[(3,7-dimethyl- | 9.7 |
| 6-octen-1-yl)oxy]acetaldehyde | |
| 3-(3,3-dimethyl-2,3-dihydro-1H- | 9.3 |
| inden-5-yl)propanal + | |
| 3-(1,1-dimethyl-2,3-dihydro-1H- | |
| inden-4-yl)propanal + | |
| 3-(1,1-dimethyl-2,3-dihydro- | |
| 1H-inden-5-yl)propanal | |
| 3-(4,4-dimethyl-1-cyclohexen- | 8.9 |
| 1-yl)propanal | |
| 3,6,7-trimethyl-2,6-octadienal | 8.7 |
| (2E)-2-methyl-3-phenyl-2-propenal | 8.1 |
| (1RS,2RS)-2,4-dimethyl-3- | 8.0 |
| cyclohexene-1-carbaldehyde + | |
| (1RS,2SR)-2,4-dimethyl-3- | |
| cyclohexene-1-carbaldehyde | |
| 10-undecenal + (9E)-9- | 7.9 |
| undecenal + (9Z)-9-undecenal | |
| 2-methylbutyl salicylate + | 7.9 |
| pentyl salicylate | |
| pine siberian oil | 7.9 |
| (1′RS,2RS,2′RS,3RS,7′RS,8′RS)- | 7.9 |
| 3,4′-dimethylspiro[oxirane-2,9′- | |
| tricyclo[6.2.1.0~2,7~]undec[4]ene] + | |
| (1′RS,2RS,2′RS,3RS,7′RS,8′RS)-3,5′- | |
| dimethylspiro[oxirane-2,9′- | |
| tricyclo[6.2.1.0~2,7~]undec[4]ene] + | |
| (1RS,2RS,4RS,6SR,8RS,9RS,11E)-11-ethylidene- | |
| 4-methyl-5-oxatetracyclo[7.2.1.0~2,8~.0~4,6~]dodecane + | |
| (1RS,2RS,4SR,6RS,8RS,9RS,10E)-10-ethylidene-4-methyl-5- | |
| oxatetracyclo[7.2.1.0~2,8~.0~4,6~]dodecane | |
| 3-(4-ethylphenyl)-2,2-dimethylpropanal + | 7.8 |
| 3-(2-ethylphenyl)-2,2-dimethylpropanal | |
| (+/−)-(3E)-3-methyl-4-(2,6,6-trimethyl-2- | 7.8 |
| cyclohexen-1-yl)-3-buten-2-one + | |
| (+/−)-(1E)-1-(2,6,6-trimethyl-2- | |
| cyclohexen-1-yl)-1-penten-3-one | |
| (+/−)-3-(4-hydroxy-4-methylpentyl)-3- | 7.8 |
| cyclohexene-1-carbaldehyde + | |
| (+/−)-4-(4-hydroxy-4-methylpentyl)- | |
| 3-cyclohexene-1-carbaldehyde | |
| (6RS,7RS)-3,5,5,6,7,8,8-heptamethyl-5,6,7,8- | 7.8 |
| tetrahydro-2-naphthalenecarbaldehyde | |
| (+/−)-8-sec-butylquinoline + | 7.8 |
| (+/−)-6-sec-butylquinoline | |
| (1RS,2RS,7SR,8RS,9Z)-9-ethylidene-3- | 7.7 |
| oxatricyclo[6.2.1.0~2,7~]undecane + | |
| (1RS,2SR,7SR,8SR, 10Z)-10-ethylidene-3- | |
| oxatricyclo [6.2.1.0~2,7~]undecane + | |
| (1RS,2RS,7SR,8RS,9E)-9-ethylidene-3- | |
| oxatricyclo[6.2.1.0~2,7~]undecane + | |
| (1RS,2SR,7SR,8SR,10E)-10-ethylidene-3- | |
| oxatricyclo [6.2.1.0~2,7~]undecane | |
| allyl (3-methylbutoxy)acetate + | 7.7 |
| (+/−)-allyl (2-methylbutoxy)acetate | |
| (2Z)-5-[(1S,3R,4R)-2,3- | 7.7 |
| dimethyltricyclo[2.2.1.0~2,6~]hept-3-yl]-2- | |
| methyl-2-penten-1-ol + | |
| (2Z)-2-methyl-5-[(1S,2R,4R)-2-methyl-3- | |
| methylenebicyclo[2.2.1]hept-2-yl]-2-penten-1-ol | |
| (Z)-3-hexenyl isobutyrate | 7.7 |
| 2,6-dimethyl-2-heptanol | 7.7 |
| (−)-(3aR,5aS,9aS,9bR)-3a,6,6,9a- | 7.7 |
| tetramethyldodecahydronaphtho[2,1-b]furan | |
| (+/−)-(2E)-2-methyl-4-(2,2,3-trimethyl-3- | 7.6 |
| cyclopenten-1-yl)-2-buten-1-ol | |
| (+/−)-3-methyl-5-phenylpentanal | 7.6 |
| (−)-(2E)-2-ethyl-4-[(1R)-2,2,3-trimethyl-3- | 7.6 |
| cyclopenten-1-yl]-2-buten-1-ol | |
| octanal | 7.5 |
| (+/−)-3-(4-isopropylphenyl)-2-methylpropanal | 7.5 |
| (+/−)-2,6-dimethyl-7-octen-2-ol | 7.5 |
| (+/−)-3-(3-isopropyl-1-phenyl)butanal | 7.5 |
| 3,5,6-trimethyl-3-cyclohexene-1-carbaldehyde + | 7.5 |
| 2,4,6-trimethyl-3-cyclohexene-1-carbaldehyde | |
| (+/−)-methyl 2,2-dimethyl-6- | 7.5 |
| methylidenecyclohexanecarboxylate | |
| 2-(6,6-dimethyl-bicyclo[3.1.1]hept- | 7.5 |
| 2-en-2-yl)ethyl acetate | |
| cabreuva oil | 7.5 |
| elemi OIL | 7.4 |
| 2-{(1RS)-1-[(1SR)-3,3- | 7.4 |
| dimethylcyclohexyl]ethoxy}-2-oxoethyl | |
| propionate + 2-{(1RS)-1-[(1RS)-3,3- | |
| dimethylcyclohexyl]ethoxy}-2-oxoethyl | |
| propionate + 2-oxo-2-{[(1RS,2RS)- | |
| 2,6,6-trimethylcycloheptyl]oxy}ethyl | |
| propionate + 2-ox0-2-{[(1RS,2SR)-2,6,6- | |
| trimethylcycloheptyl]oxy}ethyl propionate | |
| ethyl (2E,4Z)-2,4-decadienoate | 7.4 |
| methyl 2-(methylamino)benzoate | 7.4 |
| 1,1′-oxydibenzene | 7.4 |
| (+/−)-3-(4-methyl-3-penten- | 7.3 |
| 1-yl)-3-cyclohexene-1-carbaldehyde + | |
| (+/−)-4-(4-methyl-3-penten-1- | |
| yl)-3-cyclohexene-1-carbaldehyde | |
| nonanal | 7.3 |
| 1,4-dioxacycloheptadecane-5, 17-dione | 7.3 |
| (+/−)-3,7-dimethyl-6-octenal | 7.3 |
| tricyclo[5.2.1.0(2,6)]dec-3-en-8-yl propanoate + | 7.3 |
| tricyclo[5.2.1.0(2,6)]dec-4-en-8-yl propanoate | |
| (+)-(3S)-3-[(1R)-4-methyl-3-cyclohexen-1- | 7.3 |
| yl]butanal + (+)-(3R)-3-[(1R)-4-methyl- | |
| 3-cyclohexen-1-yl]butanal | |
| 3-(6,6-dimethyl-bicyclo[3.1.1]hept-2-en-2- | 7.3 |
| yl)propanal | |
| 2/3/4-(5,5,6-trimethylbicyclo[2.2.1]hept-2-yl)- | 7.3 |
| 1-cyclohexanol + | |
| 2-(1,7,7-trimethylbicyclo[2.2.1]hept-2-yl)-1-cyclohexanol | |
| (2E)-2-benzylideneoctanal | 7.2 |
| (Z)-6-nonenal | 7.2 |
| (−)-(R)-3,7-dimethyl-6-octenenitrile | 7.2 |
| cis-4-(2-methyl-2-propanyl)cyclohexyl acetate + | 7.2 |
| trans-4-(2-methyl-2-propanyl)cyclohexyl acetate | |
| 6,6-dimethoxy-2,5,5-trimethyl-2-hexene | 7.2 |
| 2,6,6-trimethyl-1,3-cyclohexadiene-1- | 7.2 |
| carbaldehyde | |
| (4E)-4-decenal | 7.2 |
| (+/−)-2-methyldecanal | 7.1 |
| (+/−)-2-methylundecanal | 7.1 |
| (+)-(R)-3,7-dimethyl-6-octenal | 7.1 |
| 2-phenylpropanal | 7.0 |
| (+/−)-2-ethyl-4-(2,2,3-trimethyl-3- | 7.0 |
| cyclopenten-1-yl)-2-buten-1-ol | |
| 7-methyl-2H-1,5-benzodioxepin-3(4H)-one | 7.0 |
| tricyclo[5.2.1.0~2,6~]dec-3-en-8-yl acetate + | 7.0 |
| tricyclo[5.2.1.0~2,6~]dec-4-en-8-yl acetate | |
| 4-(2-methoxy-2-propanyl)-1-methylcyclohexene | 7.0 |
| 1-((2RS,3RS)-2,3,8,8-tetramethyl-1,2,3,4,5,6,7,8- | 7.0 |
| octahydronaphthalen-2-yl)ethanone + | |
| 1-[(2RS,3RS,8aRS)-2,3,8,8-tetramethyl- | |
| 1,2,3,5,6,7,8,8a-octahydro-2- | |
| naphthalenyllethanone + 1-[(2RS,3RS,8aRS)- | |
| 2,3,8,8-tetramethyl-1,2,3,4,6,7,8,8a-octahydro-2- | |
| naphthalenyllethanone | |
| (+/−)-(2,5-dimethyl-2,3-dihydro-1H- | 7.0 |
| inden-2-yl)methanol | |
| (2R)-2-methyl-4-[(1R)-2,2,3-trimethyl-3- | 7.0 |
| cyclopenten-1-yl]-4-pentenal + | |
| (2S)-2-methyl-4-[(1R)-2,2,3-trimethyl- | |
| 3-cyclopenten-1-yl]-4-pentenal | |

1. An anti-dandruff composition comprising one or more perfume compounds from the following:
(A) a compound of formula (I) 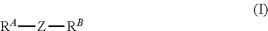
wherein
R^{A} is R₁ wherein R₁ is C₄-C₁₂ alkyl, C₄-C₅ alkenyl, or C₄-C₅ alkynyl; or 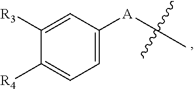
wherein R₃ is H, C₁-C₆ alkyl, or C₁-C₆ alkoxy; R₄ is H, —OH, C₁-C₆ alkyl, or C₁-C₆ alkoxy; and A is a divalent radical selected from the group consisting of a bond, C₁-C₆ alkylene, or C₂-C₆ alkenylene;
Z is C(═O) or CH₂;
R^{B} is H, OH, or OR₂, wherein R₂ is C₁₋₆ alkyl or (carbocyclyl)alkyl; and/or
(B) (2E)-2-dodecenal, (E,E)-2,4-decadienal, (E)-2-undecenal, (Z)-2-decenal, (2E)-2,4-undecadienal, (2E)-2,4-dodecadienal, (4Z)-4-dodecenal, (4Z)-4,8-dimethyl-4,9-decadienal, (4E)-4,8-dimethyl-4,9-decadienal, (Z)-4-decenal, (E)-3,7-dimethyl-2,6-octadien-1-ol, indole, methyl 2,4-dihydroxy-3,6-dimethylbenzoate, geranium rose oil, 4-methyl-2-pentylpyridine, 3-(1,3-benzodioxol-5-yl)-2-methylpropanal, 4-formyl-2-methoxyphenyl 2-methylpropanoate, [(3,7-dimethyl-6-octen-1-yl)oxy]acetaldehyde, 3-(3,3-dimethyl-2,3-dihydro-1H-inden-5-yl)propanal, 3-(1,1-dimethyl-2,3-dihydro-1H-inden-4-yl)propanal, 3-(1,1-dimethyl-2,3-dihydro-1H-inden-5-yl)propanal, 3-(4,4-dimethyl-1-cyclohexen-1-yl)propanal, 3,6,7-trimethyl-2,6-octadienal, (9E)-9-undecenal, (9Z)-9-undecenal, 2-methylbutyl salicylate, pentyl salicylate, pine siberian oil, (1′RS,2RS,2′RS,3RS,7′RS,8′RS)-3,4′-dimethylspiro[oxirane-2,9′-tricyclo[6.2.1.0~2,7~]undec[4]ene], (1′RS,2RS,2′RS,3RS,7′RS,8′RS)-3,5′-dimethylspiro[oxirane-2,9′-tricyclo[6.2.1.0~2,7~]undec[4]ene], (1RS,2RS,4RS,6SR,8RS,9RS,11E)-11-ethylidene-4-methyl-5-oxatetracyclo[7.2.1.0~2,8~.0~4,6~]dodecane, (1 RS,2RS,4SR,6RS,8RS,9RS,10E)-10-ethylidene-4-methyl-5-oxatetracyclo[7.2.1.0~2,8~.0~4,6~]dodecane, 3-(2-ethylphenyl)-2,2-dimethylpropanal, (3E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, (6RS,7RS)-3,5,5,6,7,8,8-heptamethyl-5,6,7,8-tetrahydro-2-naphthalenecarbaldehyde, 8-sec-butylquinoline, 6-sec-butylquinoline, (1 RS,2RS,7SR,8RS,9Z)-9-ethylidene-3-oxatricyclo[6.2.1.0~2,7~]undecane, (1 RS,2SR,7SR,8SR,10Z)-10-ethylidene-3-oxatricyclo[6.2.1.0~2,7~]undecane, (1 RS,2RS,7SR,8RS,9E)-9-ethylidene-3-oxatricyclo[6.2.1.0~2,7~]undecane, (1 RS,2SR,7SR,8SR,10E)-10-ethylidene-3-oxatricyclo[6.2.1.0~2,7~]undecane, allyl (3-methylbutoxy)acetate, allyl (2-methylbutoxy)acetate, (2Z)-5-[(1S,3R,4R)-2,3-dimethyltricyclo[2.2.1.0~2,6~]hept-3-yl]-2-methyl-2-penten-1-ol, (2Z)-2-methyl-5-[(1S,2R,4R)-2-methyl-3-methylenebicyclo[2.2.1]hept-2-yl]-2-penten-1-ol, (Z)-3-hexenyl isobutyrate, (3aR,5aS,9aS,9bR)-3a,6,6,9a-tetramethyldodecahydronaphtho[2,1-b]furan, (2E)-2-methyl-4-(2,2,3-trimethyl-3-cyclopenten-1-yl)-2-buten-1-ol, (2E)-2-ethyl-4-[(1R)-2,2,3-trimethyl-3-cyclopenten-1-yl]-2-buten-1-ol, 2,6-dimethyl-7-octen-2-ol, 3,5,6-trimethyl-3-cyclohexene-1-carbaldehyde, 2,4,6-trimethyl-3-cyclohexene-1-carbaldehyde, methyl 2,2-dimethyl-6-methylidenecyclohexanecarboxylate, 2-(6,6-dimethyl-bicyclo[3.1.1]hept-2-en-2-yl)ethyl acetate, cabreuva oil, elemi oil, 2-{(1 RS)-1-[(1SR)-3,3-dimethylcyclohexyl]ethoxy}-2-oxoethyl propionate, 2-{(1 RS)-1-[(1RS)-3,3-dimethylcyclohexyl]ethoxy}-2-oxoethyl propionate, 2-oxo-2-{[(1 RS,2RS)-2,6,6-trimethylcycloheptyl]oxy}ethyl propionate, 2-oxo-2-{[(1 RS,2SR)-2,6,6-trimethylcycloheptyl]oxy}ethyl propionate, ethyl (2E,4Z)-2,4-decadienoate, methyl 2-(methylamino)benzoate, 1,1′-oxydibenzene, 3-(4-methyl-3-penten-1-yl)-3-cyclohexene-1-carbaldehyde, 4-(4-methyl-3-penten-1-yl)-3-cyclohexene-1-carbaldehyde, 1,4-dioxacycloheptadecane-5,17-dione, 3,7-dimethyl-6-octenal, tricyclo[5.2.1.0(2,6)]dec-3-en-8-yl propanoate, tricyclo[5.2.1.0(2,6)]dec-4-en-8-yl propanoate, (3S)-3-[(1R)-4-methyl-3-cyclohexen-1-yl]butanal, (3R)-3-[(1R)-4-methyl-3-cyclohexen-1-yl]butanal, (6,6-dimethyl-bicyclo[3.1.1]hept-2-en-2-yl)propanal, 2/3/4-(5,5,6-trimethylbicyclo[2.2.1]hept-2-yl)-1-cyclohexanol, 2-(1,7,7-trimethylbicyclo[2.2.1]hept-2-yl)-1-cyclohexanol, (2E)-2-benzylideneoctanal, (R)-3,7-dimethyl-6-octenenitrile, cis-4-(2-methyl-2-propanyl)cyclohexyl acetate, trans-4-(2-methyl-2-propanyl)cyclohexyl acetate, 6,6-dimethoxy-2,5,5-trimethyl-2-hexene, 2,6,6-trimethyl-1,3-cyclohexadiene-1-carbaldehyde, (4E)-4-decenal, 2-methyldecanal, 2-methylundecanal, (R)-3,7-dimethyl-6-octenal, 2-ethyl-4-(2,2,3-trimethyl-3-cyclopenten-1-yl)-2-buten-1-ol, 7-methyl-2H-1,5-benzodioxepin-3(4H)-one, tricyclo[5.2.1.0~2,6~]dec-3-en-8-yl acetate, tricyclo[5.2.1.0~2,6~]dec-4-en-8-yl acetate, 4-(2-methoxy-2-propanyl)-1-methylcyclohexene, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone, (2,5-dimethyl-2,3-dihydro-1H-inden-2-yl)methanol, (2R)-2-methyl-4-[(1R)-2,2,3-trimethyl-3-cyclopenten-1-yl]-4-pentenal, and (2S)-2-methyl-4-[(1R)-2,2,3-trimethyl-3-cyclopenten-1-yl]-4-pentenal.

2. The composition of claim 1, wherein the compound of formula (I) is a selected from one or more of the following:
(i) a compound of formula (II) 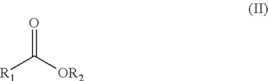
wherein
R₁ is C₄-C₅ alkyl, C₄-C₅ alkenyl, or C₄-C₅ alkynyl, and R₂ is C₁₋₆ alkyl or (carbocyclyl)alkyl;
(ii) a compound of formula (III) 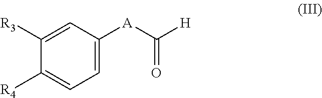
wherein
R₃ is H, C₁-C₆ alkyl, or C₁-C₆ alkoxy;
R₄ is H, OH, C₁-C₆ alkyl optionally substituted by a hydroxy group, or C₁-C₆ alkoxy;
A is a divalent radical selected from the group consisting of a bond, C₁-C₆ alkylene, or C₂-C₆ alkenylene;
(iii) a compound of formula (IV) 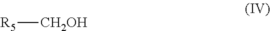
wherein R₅ is C₅-C₁₂ alkyl;
(iv) (2E,6Z)-2,6-nonadienal.

3. The composition of claim 2, wherein the compound of formula (II) is selected from: methyl octine carbonate, cyclopropylmethyl 3-hexenoate, and (+/−)-ethyl 2-methylbutanoate.

4. The composition of claim 2, wherein the compound of formula (III) is selected from: para-anisyl propanal, cyclamen propanal, (4E)-4-methyl-5-(4-methylphenyl)-4-pentenal, (4E)-2,4-dimethyl-5-(4-methylphenyl)-4-pentenal, cyclamen aldehyde, floral butanal, lilial, ethyl vanillin, 3-[4-(2-hydroxy-2-methylpropyl)phenyl]propanal, and para-anisaldehyde.

5. The composition of claim 2, wherein the compound of formula (IV) is selected from: 1-decanol, 1-dodecanol, 1-hexanol, and 2-ethyl-1-hexanol.

6. The composition of any of the previous claims, wherein the composition comprises one or more of the following compounds: p-menthan-7-ol, gamma-dodecalactone, methyl dihydrojasmonate, cinnamaldehyde, citral, lemongrass, cinnamon oil, delta-undecalactone, litsea cubeba oil, 10-undecenal, (2E,6Z)-2,6-nonadienal, (E)-3-phenyl-2-propenal, cinnamon oil ceylon, (+/−)-3-(4-methoxyphenyl)-2-methylpropanal, (2E)-2-dodecenal, (+/−)-3-(4-methoxyphenyl)-2-methylpropanal, (4E)-4-methyl-5-(4-methylphenyl)-4-pentenal, octanoic acid, (2Z)-3,7-dimethyl-2,6-octadienal, and (2E)-3,7-dimethyl-2,6-octadienal.

7. The composition of any of the previous claims, wherein the composition comprises: 1-dodecanol and 1-decanol.

8. The composition of any of claims 1 to 6, wherein the composition comprises: 1-dodecanol and p-menthan-7-ol.

9. The composition of any of claims 1 to 6, wherein the composition comprises: 1-decanol, p-menthan-7-ol, gamma-dodecalactone, methyl dihydrojasmonate and geranium rose oil.

10. The composition of any of claims 1 to 6, wherein the composition comprises: 1-dodecanol, 1-decanol, cinnamaldehyde and citral.

11. The composition of any of claims 1 to 6, wherein the composition comprises: cinnamaldehyde, citral, 10-undecenal, p-menthan-7-ol, and cyclopropylmethyl 3-hexenoate.

12. The composition of any of claims 1 to 6, wherein the composition comprises: lemongrass, cinnamon oil, and cyclopropylmethyl 3-hexenoate.

13. The composition of any of claims 1 to 6, wherein the composition comprises: (2E,6Z)-2,6-nonadienal, cinnamon oil, delta-undecalactone, and litsea cubeba oil.

14. The composition of any of claims 1 to 6, wherein the composition comprises: (2E,6Z)-2,6-nonadienal, (E)-3-phenyl-2-propenal, (+/−)-3-(4-methoxyphenyl)-2-methylpropanal, (2Z)-3,7-dimethyl-2,6-octadienal, and (2E)-3,7-dimethyl-2,6-octadienal.

15. The composition of any of claims 1 to 6, wherein the composition comprises: (2E,6Z)-2,6-nonadienal, (E)-3-phenyl-2-propenal, (2E)-2-dodecenal, (2Z)-3,7-dimethyl-2,6-octadienal, and (2E)-3,7-dimethyl-2,6-octadienal.

16. The composition of any of claims 1 to 6, wherein the composition comprises: (2E,6Z)-2,6-nonadienal, (E)-3-phenyl-2-propenal, (4E)-4-methyl-5-(4-methylphenyl)-4-pentenal, (2Z)-3,7-dimethyl-2,6-octadienal, and (2E)-3,7-dimethyl-2,6-octadienal.

17. The composition of any of claims 1 to 6, wherein the composition comprises: (2E)-2-dodecenal, (+/−)-3-(4-methoxyphenyl)-2-methylpropanal, (4E)-4-methyl-5-(4-methylphenyl)-4-pentenal, and octanoic acid.

18. The composition of any of claims 1 to 6, wherein the composition comprises: (E)-3-phenyl-2-propenal, (2E)-2-dodecenal, (+/−)-3-(4-methoxyphenyl)-2-methylpropanal, and octanoic acid.

19. The composition of any of claims 1 to 6, wherein the composition comprises: cinnamon oil ceylon, (2E)-2-dodecenal, (+/−)-3-(4-methoxyphenyl)-2-methylpropanal, and octanoic acid.

20. A hair care product comprising a composition according to any of the previous claims.

21. The hair care product of claim 20, wherein the hair care product comprises the anti-dandruff composition in an amount of from 0.05 to 5 wt. %, preferably from 0.05 to 3 wt. %, more preferably from 0.05 to 2 wt. %.

22. The hair care product of claim 20 or claim 21, wherein the hair care product is selected from the group comprising of: leave-on hair lotions, creams, and wash-off shampoos, conditioners, scalp lotion, scalp serum, scalp scrub, and shower gels.

23. The hair care product of any of claims 20 to 22, wherein the hair care product further comprises one or more anti-dandruff agent(s), preferably selected from the group consisting of hexamidine diisethionate, dichlorobenzyl alcohol (DCBA), piroctone olamine, climbazole, selenium sulphide, coal tar, azelaic acid, ketoconazole, salicylic acid, undecylenic acid, zinc pyrithione, and mixtures thereof.

24. The hair care product of any of claims 20 to 23, wherein the hair care product further comprises one or more of an Epilobium extract, saccharide isomerate, Vitamin B6 and derivatives thereof, niacinamide, and panthenol (pro-vitamin B5).

25. Use of a composition as defined in any of claims 1 to 19 or a hair care product as defined in any of claims 20-24 for the treatment or prevention of dandruff.

26. A method of treatment or prevention of dandruff, the method comprising the application to a person in need thereof a composition according to any of claims 1 to 19 or a hair care product according to any of claims 20 to 25.

27. A method of identifying a test compound or composition having use in treatment and/or prevention of dandruff, comprising
(a) assaying whether the test composition reduces Malassezia furfur growth, and/or,
(b) assaying whether the test composition reduces Malassezia globosa growth, and/or,
(c) assaying whether the test composition disrupts Malassezia restricta growth, wherein a compound or composition which is positive in one or more assays is of use in/as anti-dandruff compositions.
